(19) United States
(12) Patent Application Publication
(10) Pub. No.: US 2026/0337189 A1
(43) Pub. Date: Oct. 29, 2026

(54) Cryptolepine Salt Derivative, Preparation Method, and Use Thereof

(71) Applicant: ZHEJIANG XINNONG CHEMICAL CO., LTD., Zhejiang (CN)

(72) Inventors: Qingmin Wang, Tianjin (CN); Hongjian Song, Tianjin (CN); Pan Zhou, Tianjin (CN); Jingjing Zhang, Tianjin (CN); Yuxiu Liu, Tianjin (CN)

(21) Appl. No.: 18/857,320

(22) PCT Filed: Jul. 10, 2024
(86) PCT No.: PCT/CN2024/104657

§ 371 Date: Oct. 16, 2024

(30) Foreign Application Priority Data
Nov. 16, 2023 (CN) 202311530160.2
Nov. 16, 2023 (CN) 202311530564.1

(51) Int. Cl. C07D 471/04; A01N 43/42; A01P 1/00

(52) U.S. Cl. CPC C07D 471/04; A01N 43/42; A01P 1/00

(57) ABSTRACT

The present invention belongs to the technical field of agriculture protection and, in particular, relates to a cryptolepine salt derivative, a preparation method, and use thereof. The cryptolepine salt derivative is shown in formula (I):

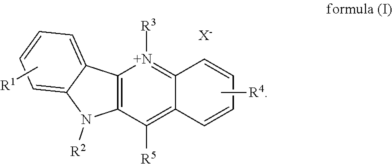

By introducing different substituents and using substituents to regulate the electricity and solubility of the cryptolepine salt derivative, the bioactivity of the cryptolepine salt derivative is improved, so that the obtained cryptolepine salt derivative has good plant virus prevention and control effect and bactericidal activity.

### CROSS REFERENCE TO RELATED APPLICATIONS

This application is a U.S. National Stage Application of International Patent Application No. PCT/CN2024/104657 filed Jul. 10, 2024, which in turn claims priority to Chinese Application No. 202311530160.2 and No. 202311530564.1, filed on Nov. 16, 2023, which is specifically and entirely incorporated by reference.

### TECHNICAL FIELD

The present invention belongs to the technical field of agriculture protection and, in particular, relates to a cryptolepine salt derivative, a preparation method and use thereof.

### BACKGROUND

In 1951, Gellert first isolated the Cryptolepine with indoloquinoline structure from Cryptolepissanguinolenta. The current study found that cryptolepine has comparable activity against Plasmodium falciparum to Chloroquine. And it has been proved that the derivative of cryptolepine have certain drug effects, specifically: 1. There have been studies to synthesize 11-position aniline indole quinoline derivative and show potential anti-tumor activity through in vivo and in vitro experiments. 2. It has been found that cryptolepine salt derivative containing aniline structure have a significant inhibitory effect on methicillin-resistant Staphylococcus aureus. Although there have been many methods for the synthesis of cryptolepine salt derivatives, there are generally shortcomings such as long synthetic route, low yield and harsh reaction conditions. And, so far, there is no use of cryptolepine salt derivatives in the prevention and control of plant viruses and sterilization.

### DISCLOSURE OF INVENTION

The purpose of the present invention is to overcome the shortcomings of the prior art and provide a cryptolepine salt derivative, preparation method and use thereof, wherein the cryptolepine salt derivative has good plant virus prevention and control effect and bactericidal activity.

In order to achieve the above-mentioned object, A second objective of the present invention is to provide a preparation method of the cryptolepine salt derivative.

In the first aspect, the present invention provides a cryptolepine salt derivative with a chemical structure shown in formula (I),

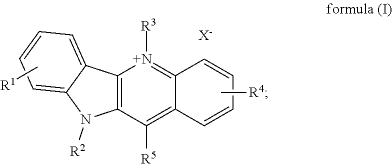

wherein
R¹ is selected from at least one group consisting of hydrogen, C1-C6 alkyl, C1-C6 alkoxy, halogen, and cyano;
R² is selected from at least one group consisting of hydrogen and protecting group;
R³ is selected from at least one group consisting of substituted or unsubstituted C1-C6 alkyl, and C6-C20 aromatic group; wherein substituent of substituted C1-C6 alkyl is selected from at least one group consisting of C3-C6 cycloalkyl;
R⁴ is selected from at least one group consisting of hydrogen, C1-C6 alkyl, C1-C6 alkoxy, halogen, C1-C6 fluoroalkyl, and C2-C5 ester group;
R⁵ is selected from at least one group consisting of hydrogen, and —NHR; wherein R is selected from at least one group consisting of substituted or unsubstituted C1-C20 alkyl, C3-C6 cycloalkyl, C3-C12 azacyclic alkyl, and C3-C12 azacyclic alkyl substituted with C1-C6 alkyl; wherein substituent of substituted C1-C20 alkyl is selected from at least one group consisting of C3-C6 cycloalkyl, amino, amino substituted with C1-C12 alkyl, C3-C12 cycloamine group, C3-C12 azacyclic alkyl, and C3-C12 azacyclic alkyl substituted with C1-C6 alkyl;
X⁻ is selected from salt-forming anion.

In the second aspect, the present invention provides a preparation method of the above-mentioned cryptolepine salt derivative, comprising:
method (1): when R⁵ is hydrogen, in presence of catalyst and formylation reagent, making compound A undergo electrophilic substitution reaction, thus to obtain the cryptolepine salt derivative;
method (2): when R⁵ is selected from —NHR, making compound B undergo nucleophilic substitution reaction with RNH₂ to obtain the cryptolepine salt derivative.

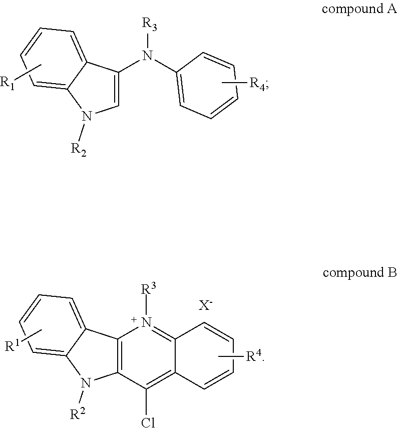

In the third aspect, the present invention provides use of the above-mentioned cryptolepine salt derivative in prevention and control of plant virus.

In the fourth aspect, the present invention provides use of the above-mentioned cryptolepine salt derivative in sterilization.

In the present invention, by introducing different substituents and using substituents to regulate the electricity and solubility of the cryptolepine salt derivative, the bioactivity of the cryptolepine salt derivative is improved, so that the obtained cryptolepine salt derivative has good plant virus prevention and control effect and bactericidal activity.

### DETAILED DESCRIPTION

The specific embodiments of the present invention are described in further detail below in conjunction with embodiments. To make the purpose, technical solution, and advantages of the present invention clearer, the technical solution in the embodiment of the present invention will be clearly and completely described below. The described embodiments are some embodiments of the present invention, not all embodiments. Based on the embodiments in the present invention, other embodiments obtained by a person skilled in the art without making creative labor belong to the scope of protection of the present invention. Unless otherwise expressly stated, throughout the description and claims, the term “including” or its transformations such as “contains” or “includes”, etc., will be understood to include the stated components and steps, without excluding the existence of other substance components or steps.

In the present invention, the dotted line in a structure indicates the bonding site and represents the bond, such as

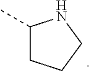

In the present invention, “substituted or unsubstituted C1-C6 alkyl” means that the alkyl has 1 to 6 carbon atoms, and the H on these carbon atoms may or may not be substituted by substituents; When substituted by substituents, the substituted H may be one or more, and when multiple H are substituted, they may be H on the same carbon atom or H on different carbon atoms. The same explanation is be appropriate for all other similar terms.

In the present invention, “C3-C12 azacyclic alkyl substituted with C1-C6 alkyl” means that the azaecyclic has 3 to 12 carbon atoms, and the H on one or more carbon atoms is substituted by alkyl with 1 to 6 carbon atoms, and when more than one H is substituted, the substituted H may be H on the same carbon atom or H on different carbon atoms. The same explanation is be appropriate for all other similar terms.

In the present invention, the alkyls in the expressions “C1-C6 alkyl” and “C1-C20 alkyl” can be either linear chain alkyl or branched chain alkyl. The same explanation is be appropriate for all other similar terms, such as the alkyl part of “C1-C6 alkoxy”, which can be linear chain or branched chain.

In the present invention, R¹ and R⁴ of structure formula

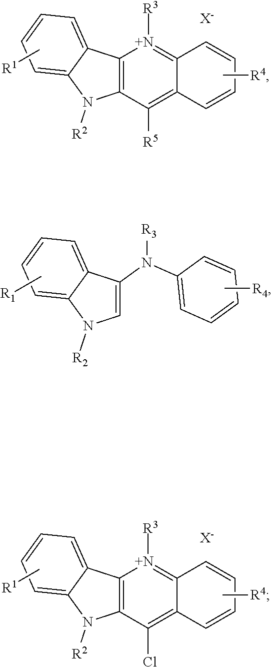

and the same, have multiple substitution sites on the benzene ring and can substitute only one site or multiple sites at the same time, for example

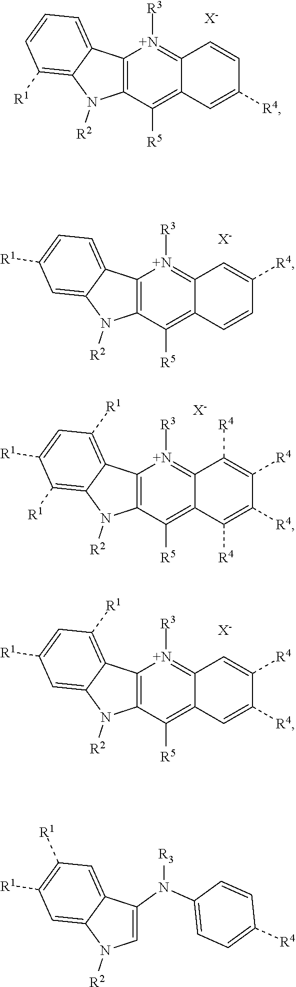

and the same, when multiple sites are substituted, each R¹ or R⁴ is independently selected group and can be the same or different.

The endpoints of ranges and any values disclosed herein are not limited to that exact range or value and should be understood to include values close to those ranges or values. For a numeric range, one or more new numeric ranges may be combined between the end values of the ranges, between the end values of the ranges and individual point values, and between individual point values, which shall be deemed to be specifically disclosed herein.

In order to better explain the technical solution, many specific details are given in the following. Those skilled in the art should understand that this technical solution can be implemented without certain specific details. In some embodiments, raw materials, methods, means, etc. well known to those skilled in the art are not described in detail in order to highlight the subject matter of the invention.

In the first aspect, the present invention provides a cryptolepine salt derivative with a chemical structure shown in formula (I),

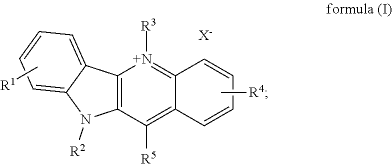

wherein
R¹ is selected from at least one group consisting of hydrogen, C1-C6 alkyl, C1-C6 alkoxy, halogen, and cyano;
R² is selected from at least one group consisting of hydrogen and protecting group;
R³ is selected from at least one group consisting of substituted or unsubstituted C1-C6 alkyl, and C6-C20 aromatic group; wherein substituent of substituted C1-C6 alkyl is selected from at least one group consisting of C3-C6 cycloalkyl;
R⁴ is selected from at least one group consisting of hydrogen, C1-C6 alkyl, C1-C6 alkoxy, halogen, C1-C6 fluoroalkyl, and C2-C5 ester group;
R⁵ is selected from at least one group consisting of hydrogen, and —NHR; wherein R is selected from at least one group consisting of substituted or unsubstituted C1-C20 alkyl, C3-C6 cycloalkyl, C3-C12 azacyclic alkyl, and C3-C12 azacyclic alkyl substituted with C1-C6 alkyl; wherein substituent of substituted C1-C20 alkyl is selected from at least one group consisting of C3-C6 cycloalkyl, amino, amino substituted with C1-C12 alkyl, C3-C12 cycloamine group, C3-C12 azacyclic alkyl, and C3-C12 azacyclic alkyl substituted with C1-C6 alkyl;
X⁻ is selected from salt-forming anion.

In the present invention, when R² is selected from the protecting group, the protecting group refers to the protecting group of amine active hydrogen suitable for the structure shown in formula (I).

In the present invention, in order to obtain a cryptolepine salt derivative having better plant virus prevention and control effect and bactericidal effect, it is necessary to further select the substituents.

According to a preferred embodiment of the present invention,
R¹ is selected from at least one group consisting of hydrogen, C1-C3 alkyl, C1-C3 alkoxy, halogen, and cyano;
R² is selected from at least one group consisting of hydrogen, p-toluenesulfonyl, benzenesulfonyl, benzyloxycarbonyl, tert-butoxycarbonyl and benzyl;
R³ is selected from at least one group consisting of substituted or unsubstituted C1-C6 alkyl, and C6-C12 aromatic group; wherein substituent of substituted C1-C6 alkyl is selected from at least one group consisting of C3-C6 cycloalkyl;
R⁴ is selected from at least one group consisting of hydrogen, C1-C3 alkyl, C1-C3 alkoxy, halogen, C1-C3 perfluoroalkyl, and C2-C5 ester group;
R⁵ is selected from at least one group consisting of hydrogen and —NHR; wherein R is selected from at least one group consisting of substituted or unsubstituted C1-C12 alkyl, C3-C6 cycloalkyl, C3-C6 azacyclic alkyl, and C3-C6 azacyclic alkyl substituted with C1-C3 alkyl; wherein substituent of substituted C1-C12 alkyl is selected from at least one group consisting of C3-C6 cycloalkyl, amino, amino substituted with C1-C6 alkyl, C3-C6 cycloamine group, C3-C6 azacyclic alkyl, and C3-C6 azacyclic alkyl substituted with C1-C3 alkyl;
X⁻ is selected from at least one group consisting of Cl⁻, Br⁻, I⁻, CH₃COO⁻, NO₃⁻, HSO₄⁻, H₂PO₄⁻, BF₄⁻, and SbF₆⁻.

In the present invention, the C2-C5 ester group can be expressed as —COOR⁰, where R⁰ is selected from methyl, ethyl, n-propyl, isopropyl, n-butyl, isobutyl and tert-butyl.

According to a preferred embodiment of the present invention, wherein,

Situation 1: when X⁻ is Cl⁻, it is satisfied as following structure:

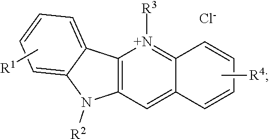

R¹ is selected from at least one group consisting of hydrogen, methyl, methoxy, F, Cl, Br, I, and cyano; R² is selected from at least one group consisting of hydrogen, p-toluenesulfonyl, and benzenesulfonyl; R³ is selected from at least one group consisting of methyl, 2,2-dimethylpropyl, cyclopropylmethyl, cyclohexylmethyl, and benzyl; R⁴ is selected from at least one group consisting of hydrogen, methyl, methoxy, F, Cl, Br, I, trifluoromethyl, and methyl formate group; R⁵ is hydrogen.

Situation 2: when X⁻ is I⁻, it is satisfied as following structure:

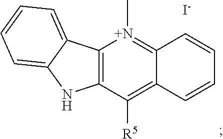

R¹, R², R⁴ is hydrogen, R³ is methyl, R⁵ is selected from at least one of —NHR, wherein R is selected from at least one group consisting of substituted or unsubstituted C1-C12 alkyl, C3-C6 cycloalkyl, C3-C6 azacyclic alkyl, and C3-C6 azacyclic alkyl substituted with C1-C3 alkyl; wherein substituent of substituted C1-C12 alkyl is selected from at least one group consisting of C3-C6 cycloalkyl, amino, amino substituted with C1-C6 alkyl, C3-C6 cycloamine group, C3-C6 azacyclic alkyl, and C3-C6 azacyclic alkyl substituted with C1-C3 alkyl.

Preferably, referring to the situation 2, when X⁻ is I⁻, it is satisfied that: R¹, R², R⁴ is hydrogen, R³ is methyl, R⁵ is selected from —NHR, wherein R comprises at least one group consisting of linear or branched C3-C12 alkyl, C3-C6 cycloalkyl, methyl substituted with C3-C6 cycloalkyl, propyl substituted with C3-C6 cycloalkyl, substituted or unsubstituted C2-C6 alkylamino, tetrahydropyrrole group, piperidyl, piperidyl methyl, n-methylpiperidyl, and n-methyl piperidine methyl; wherein substituent of substituted C2-C6 alkylamino is selected from at least one group consisting of methyl, ethyl, and

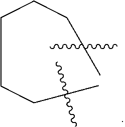 [SMILES: *CCCCC*]

For the choice of R, further preferably, R is selected from at least one group consisting of n-propyl, n-amyl, isoamyl, n-dodecyl, 2-ethylhexyl, cyclopropyl, cyclopentyl, cyclohexyl, cyclopropylmethyl, cycloamylmethyl, cyclohexylmethyl, cyclopropylethyl, cycloamylethyl, cyclohexylethyl, cyclopropylpropyl, cycloamylpropyl, cyclohexyl propyl, aminomethyl, aminoethyl, aminopropyl, aminobutyl, 2-aminopropyl, 2-methyl-2-amino-propyl, —CH₂CH₂—NH—CH₂CH₃, —CH₂CH₂CH₂—NH—CH₃, —CH₂C(CH₃)₂—CH₂—N(CH₃)₂,

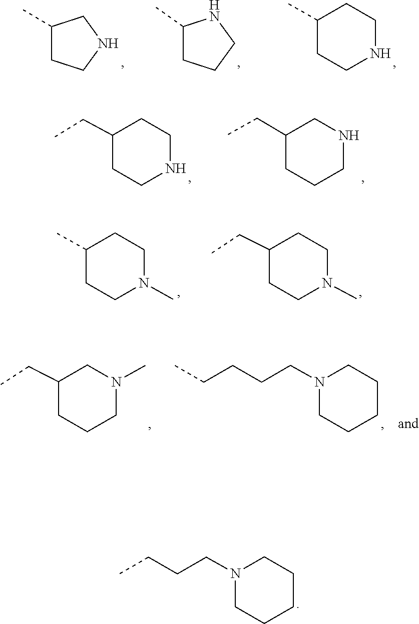

According to another preferred embodiment of the present invention, wherein, when X⁻ is Cl⁻, it is satisfied that: R¹ is selected from at least one group consisting of hydrogen, F, Cl, Br, and I; R² is selected from at least one group consisting of hydrogen, p-toluenesulfonyl, and benzenesulfonyl; R³ is selected from at least one group consisting of methyl, 2,2-dimethylpropyl, cyclopropylmethyl, cyclohexylmethyl, and benzyl; R⁴ is selected from at least one group consisting of hydrogen, methyl, Br, and trifluoromethyl; R⁵ is hydrogen.

According to another preferred embodiment of the present invention, wherein, when X⁻ is I⁻, it is satisfied that: R¹ is hydrogen, R² is hydrogen, R³ is methyl, R⁴ is hydrogen, R⁵ is selected from at least one of hydrogen and —NHR, wherein R is selected from at least one group consisting of 2-ethylhexyl, cyclopropylmethyl, aminopropyl, aminobutyl, isoamyl, 2-aminopropyl,

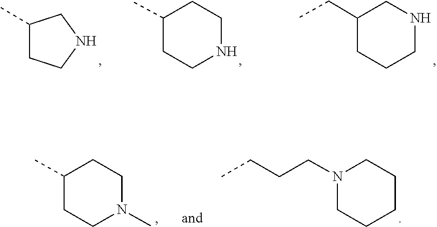

According to a further preferred embodiment of the present invention, the cryptolepine salt derivative comprises at least one of the compounds shown in formula (I-1) to formula (I-71):

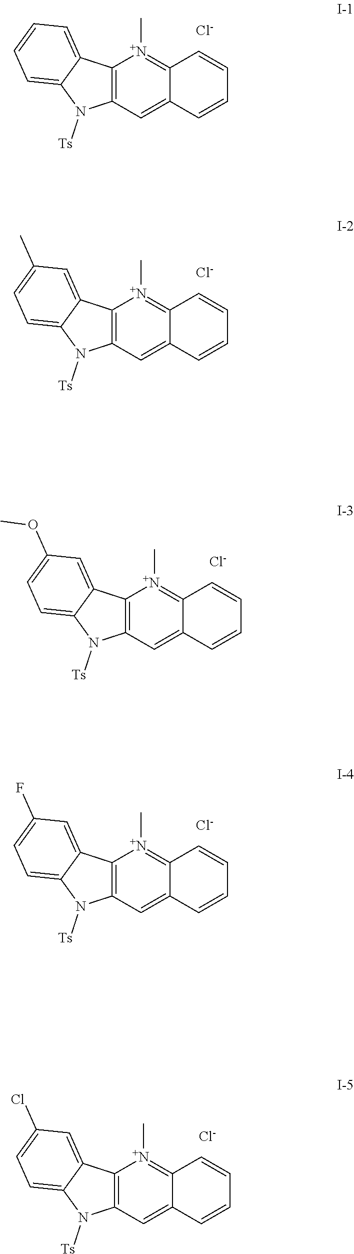

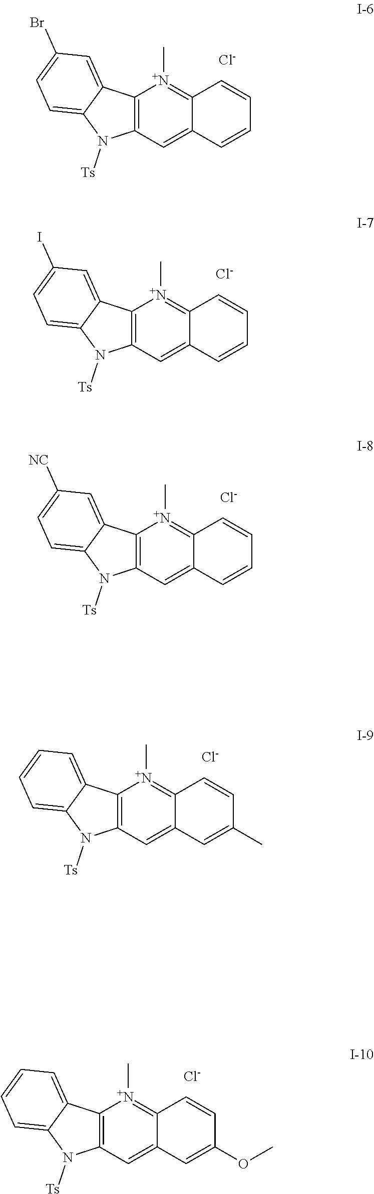

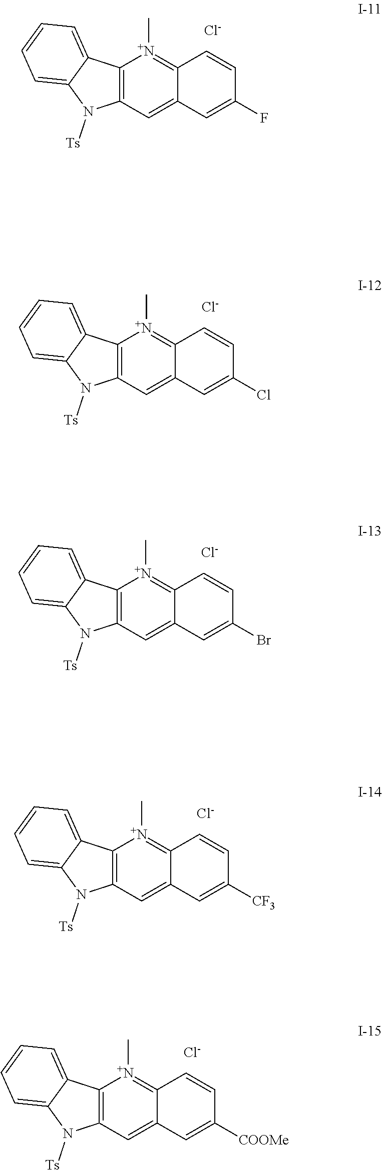

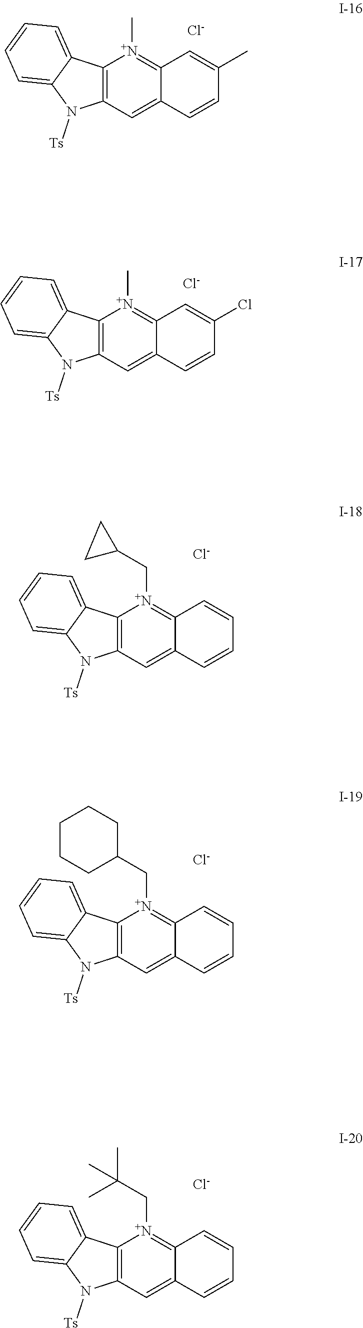

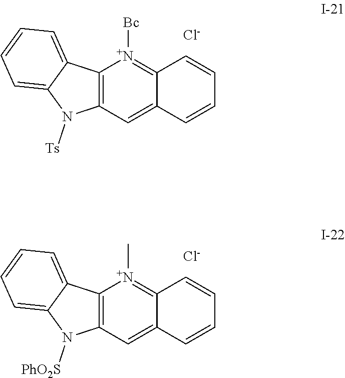

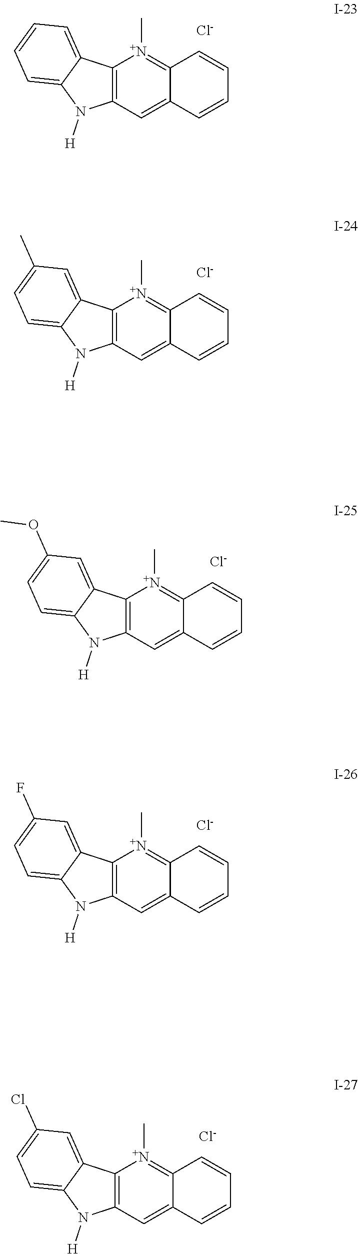

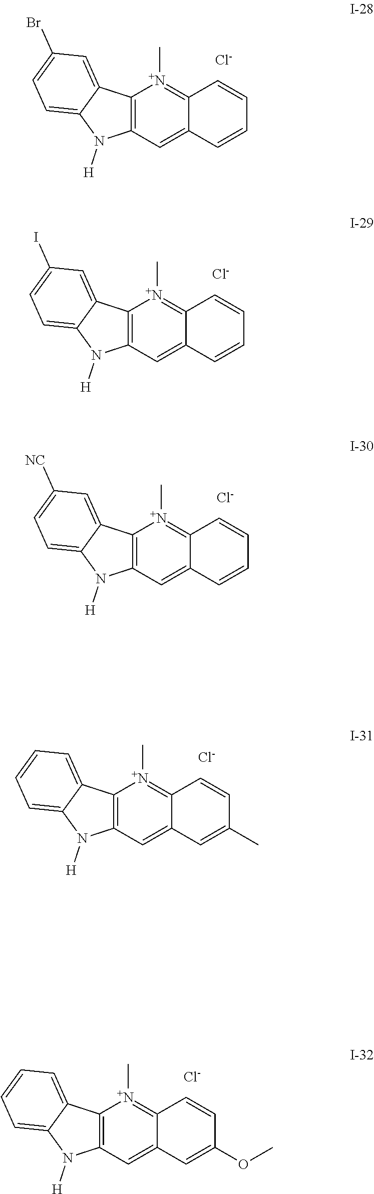

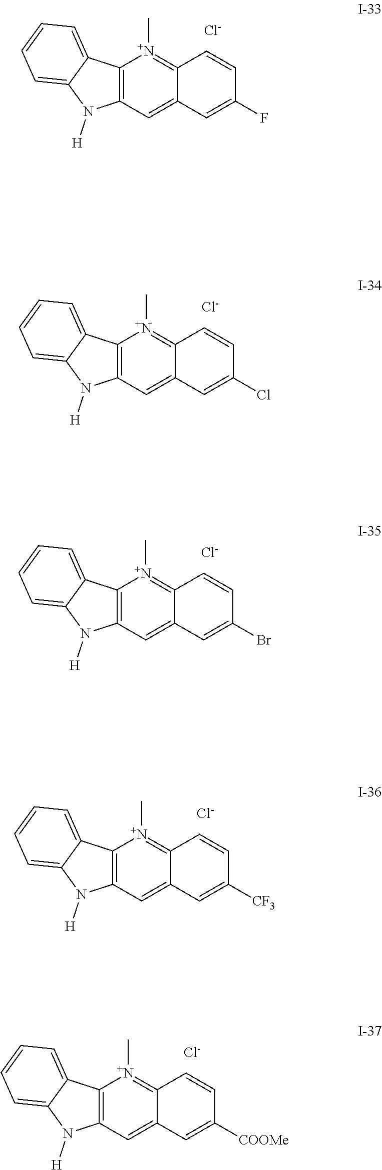

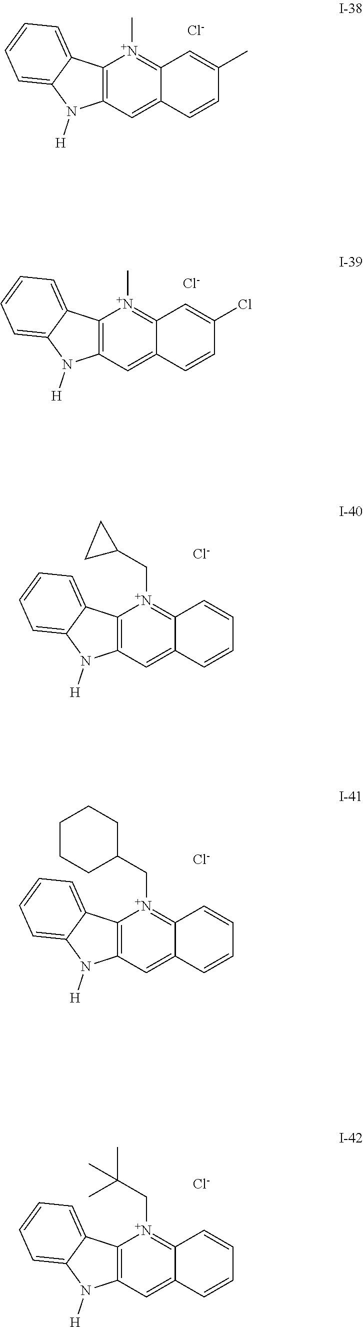

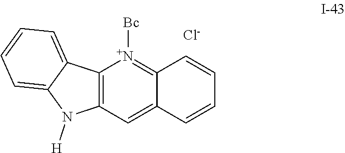

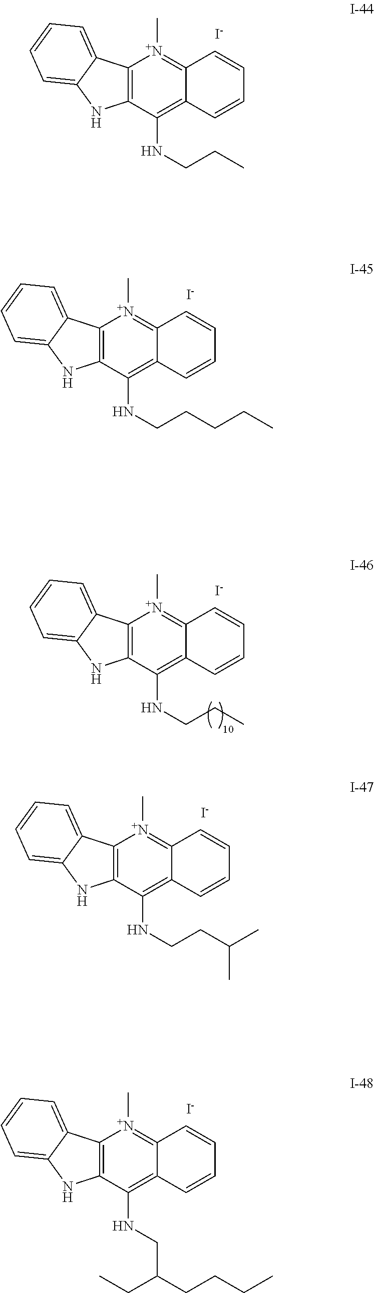

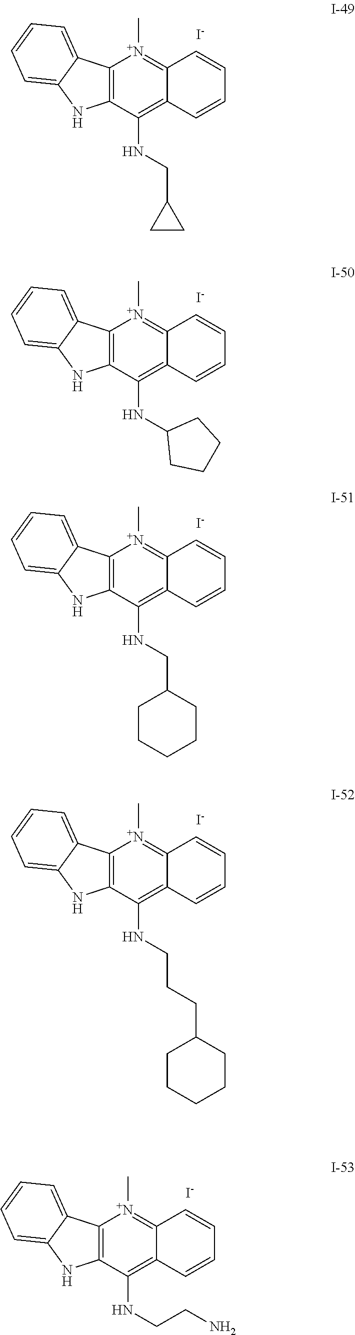

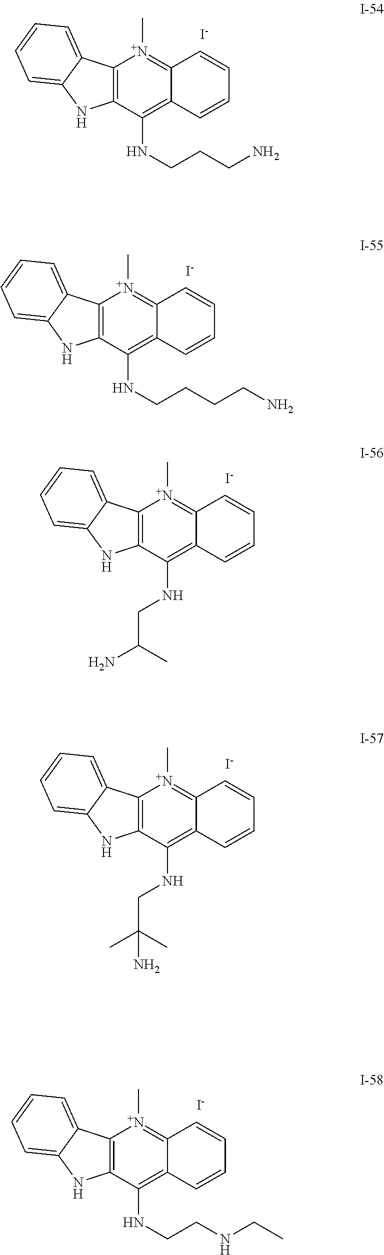

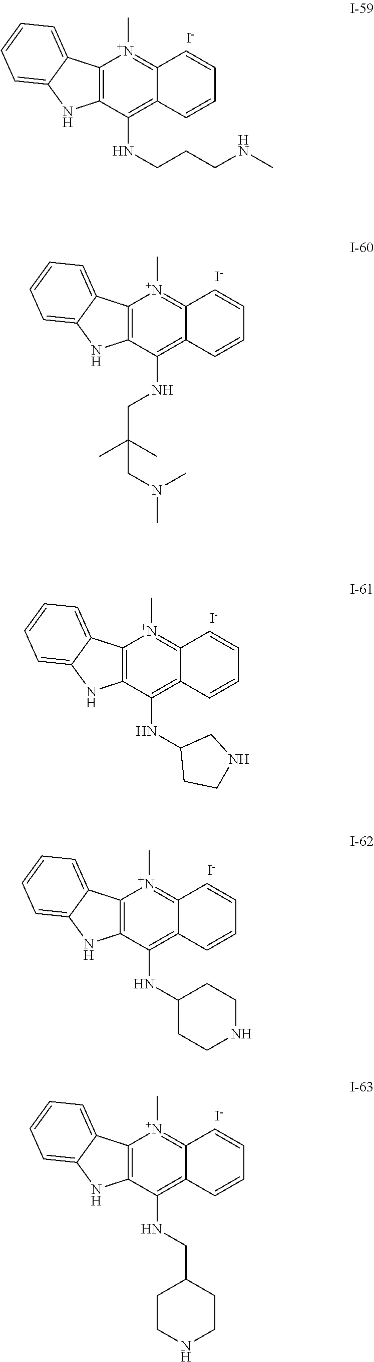

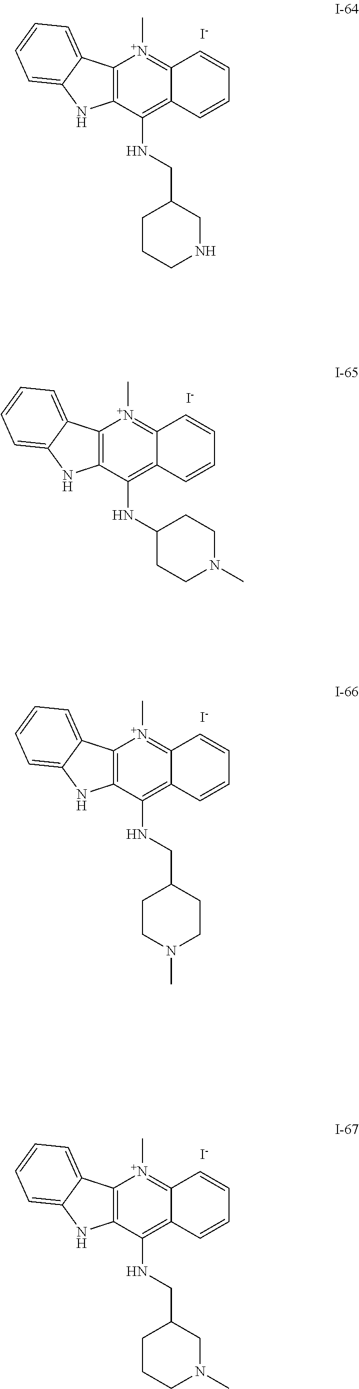

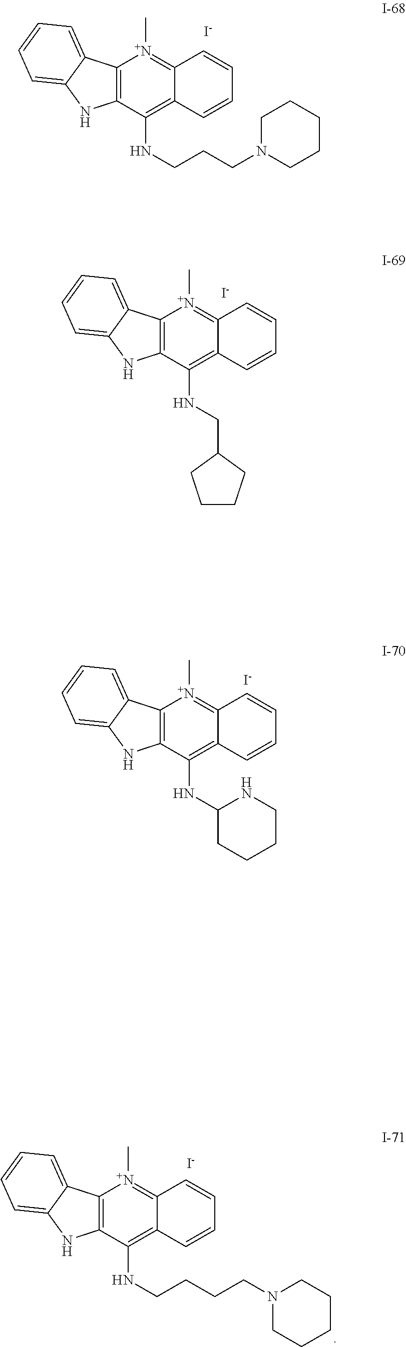

According to a particularly preferred embodiment of the present invention, wherein, the cryptolepine salt derivative comprises at least one of the following compounds:

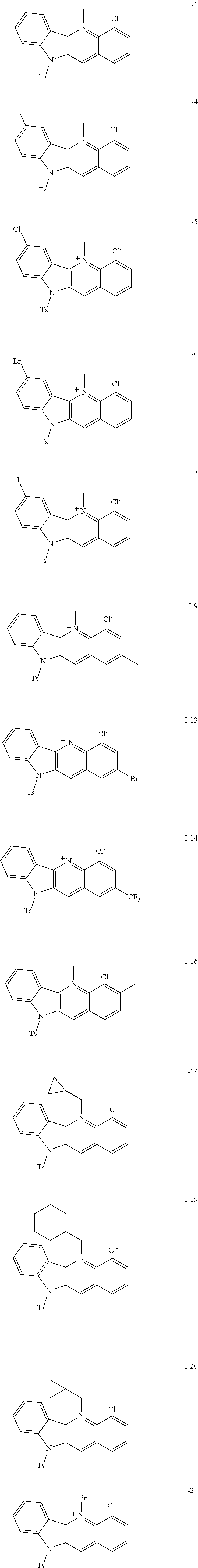

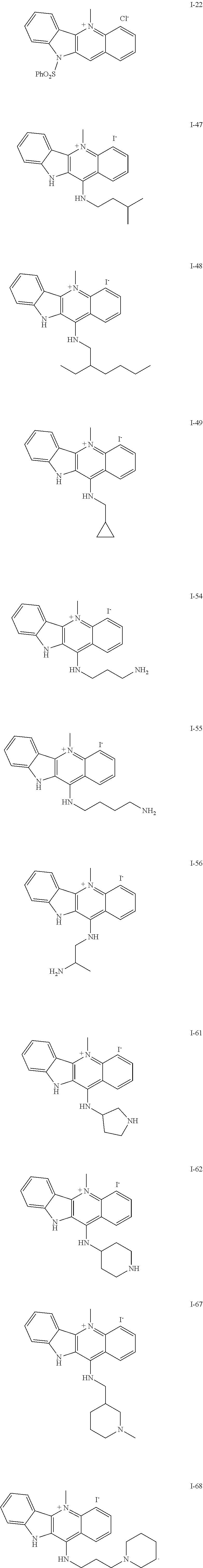

In the present invention, by introducing different substituents, the substituents can improve the virus prevention and control ability and bactericidal activity of the cryptolepine salt derivative. The cryptolepine salt derivative containing amino structure (that is when R⁵ is selected from —NHR) introduce primary amines with different substituents at the C-11 site, wherein the primary amines can be used as both hydrogen bond donors and hydrogen bond acceptors to increase the hydrogen bond site between molecule and target protein, and thus to improve the biological activity of the compound.

In the second aspect, the present invention provides a preparation method of the above-mentioned cryptolepine salt derivative, comprising:
method (1): when R⁵ is hydrogen, in presence of catalyst and formylation reagent, making compound A undergo electrophilic substitution reaction, thus to obtain the cryptolepine salt derivative;
method (2): when R⁵ is selected from —NHR, making compound B undergo nucleophilic substitution reaction with RNH₂ to obtain the cryptolepine salt derivative.

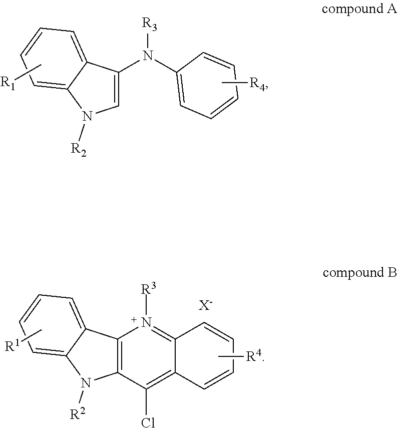

According to a preferred embodiment of the present invention, further, the preparation method of the cryptolepine salt derivative comprising:
method (1): when R⁵ is hydrogen, in presence of catalyst and formylation reagent, making compound A undergo electrophilic substitution reaction with imide salt, thus to obtain the cryptolepine salt derivative;
method (2): when R⁵ is selected from —NHR, making compound B undergo nucleophilic substitution reaction with RNH₂ under a condition of heating reflux by using ethyl acetate as solvent to obtain the cryptolepine salt derivative.

In the present invention, for the method (1), in order to make the reaction better and obtain higher product yield and purity, the reagent involved in the reaction and the reaction conditions need to be controlled.

Preferably, the catalyst is selected from at least one of POCl₃, SOCl₂, ZnCl₂ and COCl₂, preferably selected from at least one of POCl₃ and SOCl₂.

Preferably, the formylation reagent is selected from at least one of N-substituted formamides, preferably selected from at least one of N,N-Dimethylformamide, N-Methyl-N-phenylformamide, N,N-Dipropylformamide, and N-ethyl-N-methylformamide, more preferably selected from at least one of N,N-Dimethylformamide and N-Methyl-N-phenylformamide.

Preferably, molar ratio of the compound A and the catalyst is 1:1.2-3, preferably is 1:1.5-2.5, for example can be 1:1.5, 1:1.8, 1:2.2, 1:2.5, etc., and range between any of the values thereof. Relative to the above molar ratio range, when the molar ratio of compound A and catalyst is greater than 1:3, the yield of the cryptolepine salt derivative obtained is significantly decreased; when the molar ratio of compound A to catalyst was less than 1:1.2, the yield of the cryptolepine salt derivative also decreased significantly.

Preferably, the compound A is provided in solution when added to the reaction system, wherein concentration of the compound A is 0.2-1 mmol/mL, preferably 0.4-0.6 mmol/mL, for example, be values of 0.4 mmol/mL, 0.5 mmol/mL, 0.55 mmol/mL, 0.6 mmol/mL, etc., and the range between any values thereof. The solvent for dissolving compound A is at least one of the formylation reagents described above.

Preferably, temperature of the electrophilic substitution reaction is-20° C. to 30° C., preferably is −16° C. to 25° C., for example, the values of −16° C., −10° C., −5° C., 0° C., 5° C., 10° C., 20° C., 25° C., etc., and the range between any values thereof. Time of the electrophilic substitution reaction is 0.5-4 h, preferably is 1-2 h, for example, can be 1 h, 1.5 h, 1.8 h and 2 h, and other values and the range between any values thereof.

According to the present invention, the imide salt is an intermediate

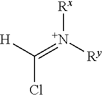

obtained by a reaction of the catalyst and the formylation reagent, wherein R^{x} and R^{y} are two substituents on the N position of N-substituted formamide, respectively.

In the present invention, for the method (2), in order to make the reaction better and obtain a higher product yield and purity, the reaction conditions need to be controlled.

Preferably, temperature of the heating reflux is 90-150° C., preferably is 110-130° C., for example, can be 110° C., 115° C., 122° C., 128° C., etc. and the range between any values thereof. Time of the heating reflux is 10-24 h, preferably is 15-17 h, for example, can be 15 h, 16 h, 16.5 h, 17 h and other values and the range between any values thereof.

Preferably, molar ratio of the compound B to RNH₂ is 1:1-5, preferably is 1:1-3, for example, the values of 1:1, 1:2, 1:2.5, and 1:3, and other values and the range between any values thereof.

In the third aspect, the present invention provides use of the above-mentioned cryptolepine salt derivative in prevention and control of plant virus.

In the fourth aspect, the present invention provides use of the above-mentioned cryptolepine salt derivative in sterilization.

The cryptolepine salt derivative provided by the present invention has excellent anti-plant virus activity, and the cryptolepine salt derivative shown in formula (I) exhibits good anti-tobacco Mosaic virus activity. In addition, the cryptolepine salt derivative provided by the invention also has high bactericidal activity, especially against the pathogens causing powdery mildew of cucumber and rice sheath blight disease.

The invention is described in detail in the following embodiments, but the invention is not limited to the following embodiments, the reagents are not specifically stated in the embodiments, and can be obtained by purchase.

In the following examples:

Compounds of examples 1-22 are prepared by using the above method (1), while compounds of examples 23-47 are prepared by using the above method (2).

Compound A is prepared according to the method described in the literature (Tetrahedron Lett. 1998, 39:6465-6466.).

Compound B is prepared according to the method described in the literature (J. Med. Chem. 1998, 41 (15): 2754-2764; J. Ethnopharmacol. 2005, 100 (1-2): 67-71).

### Preparation Examples 1-22

Preparation Examples 1-22 are used to illustrate the preparation method of compound A.

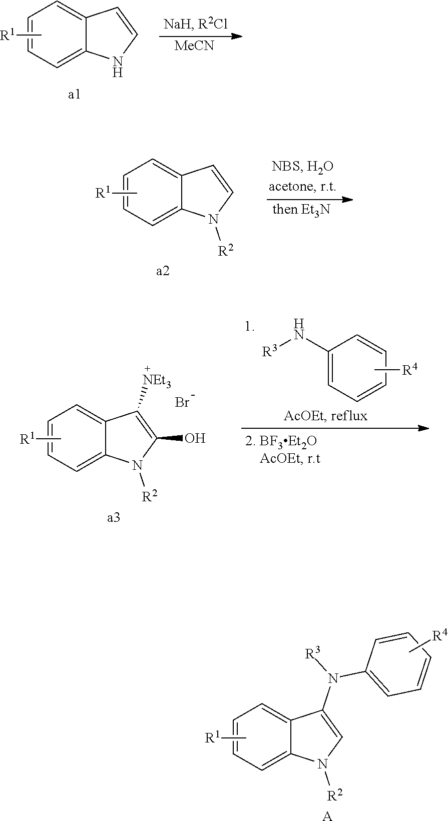

30 mmol of compound a1 (each compound a1 selected for preparation examples 1-22 is shown in Table 1) was dissolved in 30 mL acetonitrile, after slowly adding 40 mmol of sodium hydride in batches at 0° C., the reaction system was stirred for 10 min and then to return to room temperature, after adding 33 mmol R²Cl (each compound R²Cl selected for preparation examples 1-22 is shown in Table 1) and until the reaction was completed, the saturated ammonium chloride aqueous solution was added for quenching the reaction, then the reaction system was extracted with ethyl acetate, the obtained organic phase was combined, washed by saturated salt water, dried with anhydrous sodium sulfate, distilled under reduced pressure, and purified by column chromatography to obtain white solid compound a2.

11 mmol of compound a2 and 110 mmol of high purity water were dissolved in 110 mL of acetone, then 12 mmol of NBS (N-Bromosuccinimide) was added until the reaction was completed, then 12 mmol of triethylamine was added to the reaction system and stirred for 1 h to precipitate a large amount of white solid, then the reaction system was suction filtered, the obtained solid was washed with acetone several times and dried to get compound a3.

5 mmol of compound a3, 5.5 mmol of

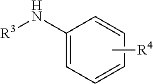

(the specific compound selected for preparation examples 1-22 is shown in Table 1) and 10 mmol of triethylamine were dissolved in 100 mL of ethyl acetate, then the mixture was heated and refluxed for 6 h, and after the reaction was completed, water was added to the reaction system which was latter extracted by ethyl acetate, the obtained organic phase was combined, washed by saturated salt water, dried with anhydrous sodium sulfate. After vacuum distillation, it was dissolving in ethyl acetate, after adding 25 mmol of boron trifluoride ether solution, it was reacted at 50° C. for 3 h and cooled to room temperature after reaction was completed, the reaction system was slowly added with saturated sodium bicarbonate solution, extracted by ethyl acetate, the obtained organic phase was combined, washed by saturated salt water, dried with anhydrous sodium sulfate, distilled under reduced pressure and purified by column chromatography to obtain white solid compound A (see Table 1 for the compound A obtained in preparation examples 1-22 respectively).

TABLE 1

| serial number | Compound a1 | R²Cl | 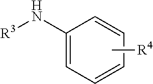 | Compound A |
|---|---|---|---|---|
| Preparation Example 1 | Indole | P-toluenesulfonyl chloride | 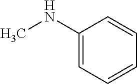 | 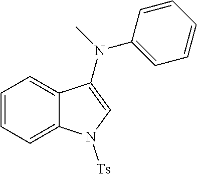 [SMILES: Cc1ccc(S(=O)(=O)n2cc(N(C)c3ccccc3)c3ccccc32)cc1] |
| Preparation Example 2 | 5-methylindole | P-toluenesulfonyl chloride | 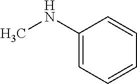 | 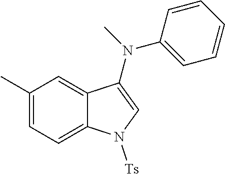 [SMILES: Cc1ccc(S(=O)(=O)n2cc(N(C)c3ccccc3)c3cc(C)ccc32)cc1] |
| Preparation Example 3 | 5-methoxyindole | P-toluenesulfonyl chloride | 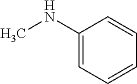 | 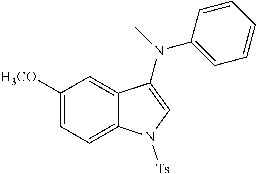 [SMILES: COc1ccc2c(c1)c(N(C)c1ccccc1)cn2S(=O)(=O)c1ccc(C)cc1] |
| Preparation Example 4 | 5-F-indole | P-toluenesulfonyl chloride | 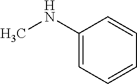 | 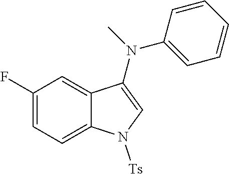 [SMILES: Cc1ccc(S(=O)(=O)n2cc(N(C)c3ccccc3)c3cc(F)ccc32)cc1] |
| Preparation Example 5 | 5-Cl-indole | P-toluenesulfonyl chloride | 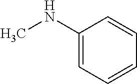 | 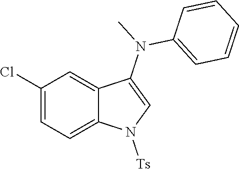 [SMILES: Cc1ccc(S(=O)(=O)n2cc(N(C)c3ccccc3)c3cc(Cl)ccc32)cc1] |
| Preparation Example 6 | 5-Br-indole | P-toluenesulfonyl chloride | 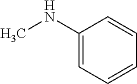 | 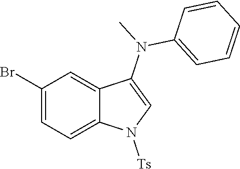 [SMILES: Cc1ccc(S(=O)(=O)n2cc(N(C)c3ccccc3)c3cc(Br)ccc32)cc1] |
| Preparation Example 7 | 5-I-indole | P-toluenesulfonyl chloride | 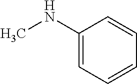 | 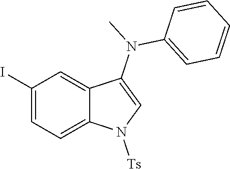 [SMILES: Cc1ccc(S(=O)(=O)n2cc(N(C)c3ccccc3)c3cc(I)ccc32)cc1] |
| Preparation Example 8 | 5-cyanoindole | P-toluenesulfonyl chloride | 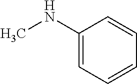 | 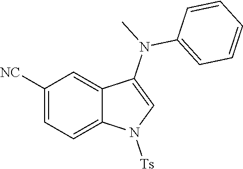 [SMILES: Cc1ccc(S(=O)(=O)n2cc(N(C)c3ccccc3)c3cc(C#N)ccc32)cc1] |
| Preparation Example 9 | Indole | P-toluenesulfonyl chloride | 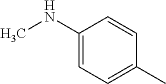 | 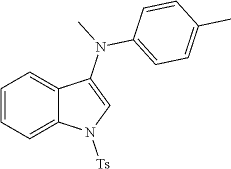 [SMILES: Cc1ccc(N(C)c2cn(S(=O)(=O)c3ccc(C)cc3)c3ccccc23)cc1] |
| Preparation Example 10 | Indole | P-toluene sulfonyl chloride | 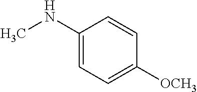 | 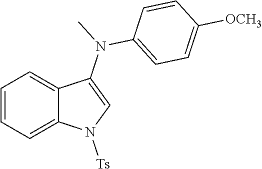 [SMILES: COc1ccc(N(C)c2cn(S(=O)(=O)c3ccc(C)cc3)c3ccccc23)cc1] |
| Preparation Example 11 | Indole | P-toluenesulfonyl chloride | 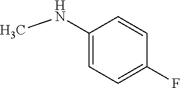 | 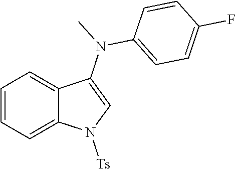 [SMILES: Cc1ccc(S(=O)(=O)n2cc(N(C)c3ccc(F)cc3)c3ccccc32)cc1] |
| Preparation Example 12 | Indole | P-toluenesulfonyl chloride | 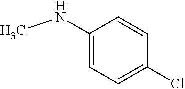 | 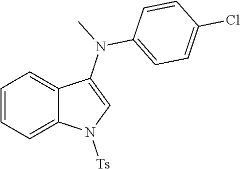 [SMILES: Cc1ccc(S(=O)(=O)n2cc(N(C)c3ccc(Cl)cc3)c3ccccc32)cc1] |
| Preparation Example 13 | Indole | P-toluenesulfonyl chloride | 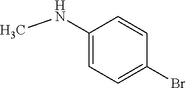 | 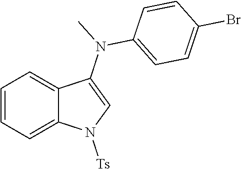 [SMILES: Cc1ccc(S(=O)(=O)n2cc(N(C)c3ccc(Br)cc3)c3ccccc32)cc1] |
| Preparation Example 14 | Indole | P-toluenesulfonyl chloride | 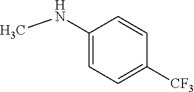 | 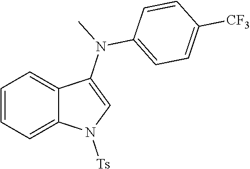 [SMILES: Cc1ccc(S(=O)(=O)n2cc(N(C)c3ccc(C(F)(F)F)cc3)c3ccccc32)cc1] |
| Preparation Example 15 | Indole | P-toluenesulfonyl chloride | 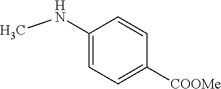 | 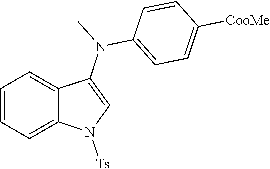 |
| Preparation Example 16 | Indole | P-toluenesulfonyl chloride | 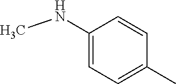 | 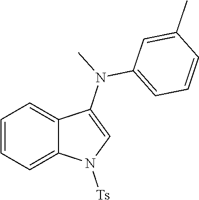 [SMILES: Cc1ccc(S(=O)(=O)n2cc(N(C)c3cccc(C)c3)c3ccccc32)cc1] |
| Preparation Example 17 | Indole | P-toluenesulfonyl chloride | 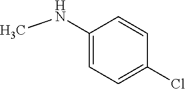 | 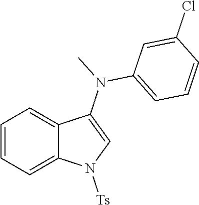 [SMILES: Cc1ccc(S(=O)(=O)n2cc(N(C)c3cccc(Cl)c3)c3ccccc32)cc1] |
| Preparation Example 18 | Indole | P-toluenesulfonyl chloride | 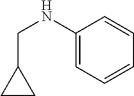 [SMILES: c1ccc(NCC2CC2)cc1] | 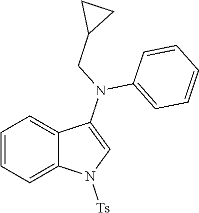 [SMILES: Cc1ccc(S(=O)(=O)n2cc(N(CC3CC3)c3ccccc3)c3ccccc32)cc1] |
| Preparation Example 19 | Indole | P-toluenesulfonyl chloride | 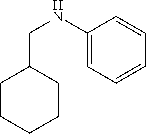 [SMILES: c1ccc(NCC2CCCCC2)cc1] | 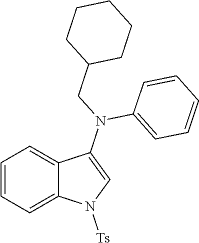 [SMILES: Cc1ccc(S(=O)(=O)n2cc(N(CC3CCCCC3)c3ccccc3)c3ccccc32)cc1] |
| Preparation Example 20 | Indole | P-toluenesulfonyl chloride | 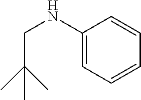 | 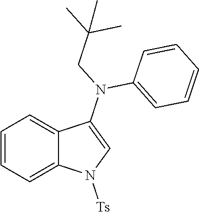 [SMILES: Cc1ccc(S(=O)(=O)n2cc(N(CC(C)(C)C)c3ccccc3)c3ccccc32)cc1] |
| Preparation Example 21 | indole | P-toluenesulfonyl chloride | 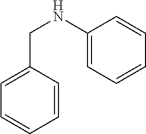 [SMILES: c1ccc(CNc2ccccc2)cc1] | 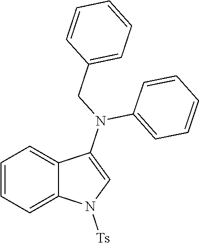 [SMILES: Cc1ccc(S(=O)(=O)n2cc(N(Cc3ccccc3)c3ccccc3)c3ccccc32)cc1] |
| Preparation Example 22 | Indole | Benzene sulfonyl chloride | 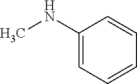 | 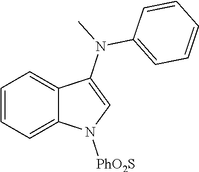 |

### Preparation Example 23

Preparation Example 23 is used to illustrate the preparation method of compound B.

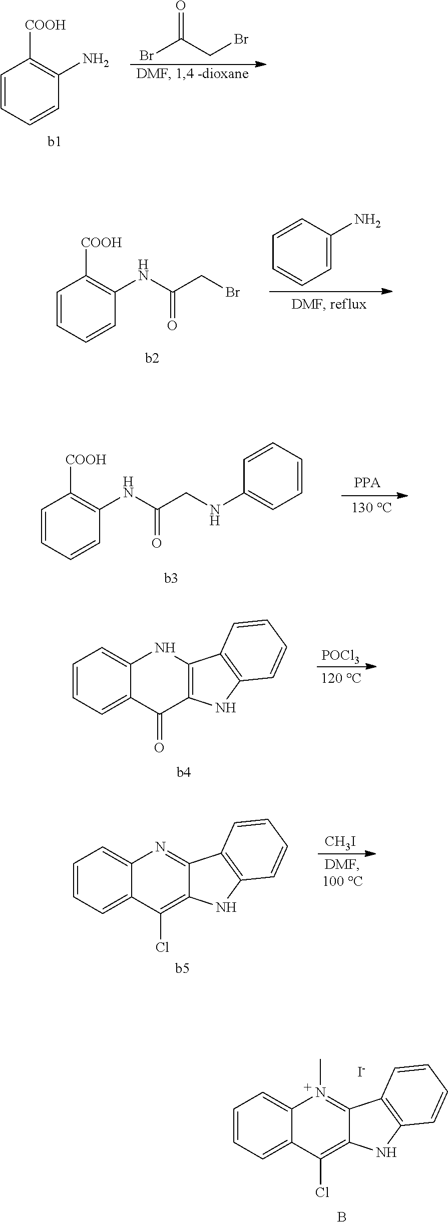

20 mmol of compound b1 was dissolved in a mixture solution of 8 mL N, N dimethylformamide and 8 mL 1,4-dioxane, after slowly dripped with 24 mmol of bromoacetyl bromide at 0° C., the reaction system was stirred overnight at room temperature. After the reaction is completed, the reaction liquid was poured into an appropriate amount of ice water, a large amount of white solid was precipitated, suction filtered, washed five times with a large amount of water, and dried to obtain the compound b2.

19.4 mmol of compound b2 was dissolved in 10 mL of N,N dimethylformamide, after adding 69.4 mmol of aniline, the reaction system was heated and refluxed at 120° C. for 18 h, then cooled to room temperature after the reaction was completed, by slowly adding 5% potassium hydroxide solution to adjust pH to 11, the reaction system was extracted with methylene chloride several times, the obtained water phase was combined and pH of which was adjusted to 2-3 with 5% hydrobromic acid solution and then standed at room temperature overnight, a large amount of white solid was precipitated, suction filtered, and dried to obtain compound b3.

150 g PPA (polyphosphates) was added to 15.8 mmol of compound b3, the mixture was heated at 130° C. for 2 h, cooled to room temperature after the reaction was completed, the reaction liquid was poured into an appropriate amount of ice water, pH of which was adjusted to neutral with saturated potassium hydroxide solution, extracted by ethyl acetate while the organic phase was combined, washed with saturated salt water, dried with anhydrous sodium sulfate, distilled under reduced pressure and purified by column chromatography to obtain compound b4.

33 mL POCl₃ (phosphorus oxychloride) was slowly dripped to 13 mmol of compound b4, the mixture was heated and reflexed for 2 h at 120° C., cooled to room temperature after the reaction was completed, the reaction liquid was slowly poured into appropriate amount of ice water, pH of which was adjusted to neutral with saturated potassium hydroxide solution, extracted by ethyl acetate while the organic phase was combined, and washed with saturated salt water, dried with anhydrous sodium sulfate, distilled under reduced pressure and purified by column chromatography to obtain compound b5.

0.5 mmol of compound b5 was dissolved in 2 mL N, N dimethylformamide, after adding 1.5 mmol of iodomethane, the mixture was heated at 100° C. for 8 h, then cooled to room temperature after the reaction was completed, though adding ethyl acetate to the mixture, a large amount of solid precipitated, suction filtered, washed with ethyl acetate several times, and dried to obtain compound B.

### Examples 1-22

Examples 1-22 are used to illustrate the synthesis of the cryptolepine salt derivatives shown in formulas (I-1) to (I-22).

2 mmol POCl₃ (phosphorus oxytrichloride) was slowly dripped into 1 mL of N, N dimethylformamide at −16° C. and stirred for 0.5 h. At 0° C., 1 mmol of the corresponding compound A (Examples 1-22 respectively adopt compound A obtained from preparation example 1-22 above) dissolved in 2 mL N, N dimethylformamide was further added into the above solution and the obtained mixture was reacted at room temperature for 1 h. After the reaction was completed, the reaction liquid was poured into ice water, stirred to precipitate the solid, and 10% sodium hydroxide solution was added to adjust the pH to about 10. After standing for 1 h, the reaction system was filtered to obtain the cryptolepine salt derivatives as shown in formula (I-1) to Formula (I-22) respectively (the yield, melting point and appearance are detailed in Table 3).

### Examples 23-47

Examples 23-47 are used to illustrate the synthesis of the cryptolepine salt derivatives shown in formulas (I-44) to (I-68).

0.5 mmol of compound B was weighed and dissolved in 20 mL ethyl acetate, 1 mmol of corresponding RNH₂ (group R of compound RNH₂ selected for example 23-47 is shown in Table 2) was added into the above solution and the obtained mixture was heated and refluxed for 16 h. After the reaction was completed, it was cooled to room temperature, some solids were precipitated, suction filtered, washed with ethyl acetate several times, and dried to obtain the cryptolepine salt derivatives as shown in formula (I-44) to formula (I-68) respectively (the yield, melting point and appearance are detailed in Table 3).

TABLE 2

| Example serial number | R |
|---|---|
| Example 23 | n-propyl |
| Example 24 | n-amyl |
| Example 25 | n-dodecyl |
| Example 26 | isoamyl |
| Example 27 | 2-ethylhexyl |
| Example 28 | cyclopropylmethyl |
| Example 29 | cycloamylmethyl |
| Example 30 | cyclohexylmethyl |
| Example 31 | cyclohexylpropyl |
| Example 32 | aminoethyl |
| Example 33 | aminopropyl |
| Example 34 | aminobutyl |
| Example 35 | 2-aminopropyl |
| Example 36 | 2-methyl-2-amino-propyl |
| Example 37 | —CH₂CH₂—NH—CH₂CH₃ |
| Example 38 | —CH₂CH₂CH₂—NH—CH₃ |
| Example 39 | —CH₂C(CH₃)₂—CH₂—N(CH₃)₂ |
| Example 40 | 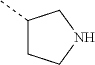 |
| Example 41 | 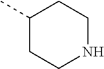 |
| Example 42 | 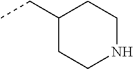 |
| Example 43 | 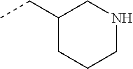 |
| Example 44 | 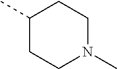 |
| Example 45 | 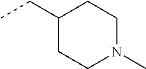 |
| Example 46 | 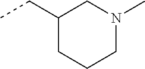 |
| Example 47 | 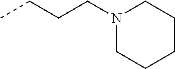 |

TABLE 3

| Example | | | Product | |
|---|---|---|---|---|
| serial | Corresponding | Product | melting | |
| number | compound | yield/% | point/° C. | Product status |
| Example 1 | Formula (I-1) | 93 | 148-149 | Yellow solid |
| Example 2 | Formula (I-2) | 92 | 151-152 | Yellow solid |
| Example 3 | Formula (I-3) | 99 | 162-163 | Bright orange solid |
| Example 4 | Formula (I-4) | 86 | 160-161 | Bright yellow solid |
| Example 5 | Formula (I-5) | 99 | 158-159 | Bright yellow solid |
| Example 6 | Formula (I-6) | 94 | 154-155 | Yellow solid |
| Example 7 | Formula (I-7) | 92 | 138-139 | Orange-yellow solid |
| Example 8 | Formula (I-8) | 92 | 112-113 | Pale yellow solid |
| Example 9 | Formula (I-9) | 97 | 152-153 | Yellow-green solid |
| Example 10 | Formula (I-10) | 99 | 188-189 | Yellow solid |
| Example 11 | Formula (I-11) | 93 | 153-154 | Yellow solid |
| Example 12 | Formula (I-12) | 87 | 178-179 | Green solid |
| Example 13 | Formula (I-13) | 95 | 167-168 | Yellow solid |
| Example 14 | Formula (I-14) | 98 | 165-166 | Bright yellow solid |
| Example 15 | Formula (I-15) | 91 | 157-158 | Yellow-green solid |
| Example 16 | Formula (I-16) | 99 | 141-142 | Yellow-green solid |
| Example 17 | Formula (I-17) | 94 | 154-155 | Yellow solid |
| Example 18 | Formula (I-18) | 99 | 113-114 | Yellow solid |
| Example 19 | Formula (I-19) | 98 | 142-143 | Yellow solid |
| Example 20 | Formula (I-20) | 95 | 127-128 | Yellow solid |
| Example 21 | Formula (I-21) | 99 | 156-157 | Yellow solid |
| Example 22 | Formula (I-22) | 97 | 140-141 | Bright yellow solid |
| Example 23 | Formula (I-44) | 59 | Greater | Yellow-green solid |
| | | | than 250 | |
| Example 24 | Formula (I-45) | 60 | 230-231 | Yellow solid |
| Example 25 | Formula (I-46) | 57 | 171-172 | Yellow solid |
| Example 26 | Formula (I-47) | 78 | 238-239 | Yellow solid |
| Example 27 | Formula (I-48) | 72 | 200-201 | Yellow solid |
| Example 28 | Formula (I-49) | 62 | Greater | Yellow solid |
| | | | than 250 | |
| Example 29 | Formula (I-50) | 60 | 248-249 | Brown-yellow solid |
| Example 30 | Formula (I-51) | 79 | 243-244 | Yellow solid |
| Example 31 | Formula (I-52) | 51 | 234-235 | Yellow solid |
| Example 32 | Formula (I-53) | 87 | 235-236 | Yellow solid |
| Example 33 | Formula (I-54) | 86 | 198-199 | Yellow solid |
| Example 34 | Formula (I-55) | 84 | 203-204 | Yellow solid |
| Example 35 | Formula (I-56) | 87 | 221-222 | Yellow solid |
| Example 36 | Formula (I-57) | 89 | 252-253 | Yellow solid |
| Example 37 | Formula (I-58) | 49 | 233-234 | Yellow solid |
| Example 38 | Formula (I-59) | 74 | 187-188 | Tangerine solid |
| Example 39 | Formula (I-60) | 64 | 242-243 | Yellow solid |
| Example 40 | Formula (I-61) | 79 | 184-185 | Brown-yellow solid |
| Example 41 | Formula (I-62) | 81 | 245-246 | Orange solid |
| Example 42 | Formula (I-63) | 78 | 217-218 | Yellow solid |
| Example 43 | Formula (I-64) | 82 | 200-201 | Yellow solid |
| Example 44 | Formula (I-65) | 85 | 240-241 | Yellow-green solid |
| Example 45 | Formula (I-66) | 75 | 246-247 | Yellow solid |
| Example 46 | Formula (I-67) | 71 | 177-178 | Yellow solid |
| Example 47 | Formula (I-68) | 73 | 208-209 | Yellow solid |

### Reference Example 1

Synthesis of 5-methyl-7-bromo 5H-indolo[3,2-b]quinoline (structure of which is shown in the following formula (II)).

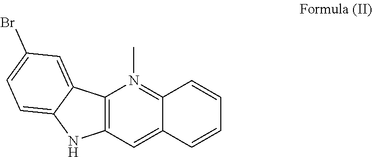

2 mmol POCl₃ (phosphorus oxychloride) was dripped to 1 mL N,N-dimethylformamide at −16° C. and stirred for 0.5 h, and then

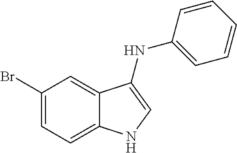 [SMILES: Brc1ccc2[nH]cc(Nc3ccccc3)c2c1]

was added to the above solution at room temperature and stirred for 1 h. Then saturated sodium bicarbonate solution was added to the reaction solution, the obtained reaction solution was extracted with ethyl acetate, organic layer of which was washed with saturated sodium bicarbonate solution, and the organic phase was combined, dried with magnesium sulfate and concentrated under reduced pressure to obtain solid residue. Using dichloromethane/methanol as eluent, the yellow solid product was purified by column chromatography.

0.1 mol of dimethylamine hydrochloride was dissolved in 1 mL N,N-dimethylformamide, then 0.05 mol of the yellow solid product obtained in the previous step was added to the reaction system, the obtained reaction system was heated and refluxed for 1.5 h, cooled after the reaction was completed, after adding 5 mL of 5% sodium carbonate solution, the reaction system was stirred at room temperature for 10 min, extracted with ethyl acetate for 3 times, and then washed with saturated sodium carbonate. The organic phase was combined, dried with magnesium sulfate and concentrated under reduced pressure to concentrate solid residue. The purple solid product was purified by column chromatography with methylene chloride/methanol as eluent, and the yield was 35%.

The results of structural characterization were as follows:

¹HNMR (400 MHZ, DMSO-d6) δ9.05 (s, 1H), 8.70 (s, 1H), 8.58 (d, J=9.0 Hz, 1H), 8.44 (d, J=8.3 Hz, 1H), 8.02-7.87 (m, 1H), 7.74 (t, J=7.5 Hz, 1H), 7.69-7.60 (m, 2H), 4.92 (s, 3H).

### Reference Example 2

Synthesis of 11-(4′-(2-aminoethyl)-piperazinyl) substituted cryptolepine iodide salt (structure of which is shown in the following formula (III)).

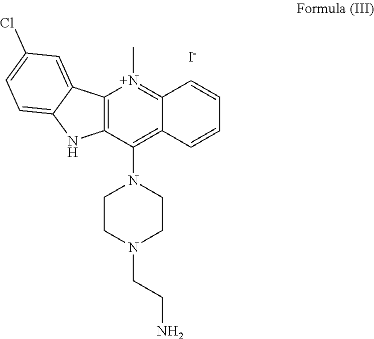 [SMILES: C[n+]1c2ccccc2c(N2CCN(CCN)CC2)c2[nH]c3ccc(Cl)cc3c21 ; [I-]]

0.1 mol of 7-chloro-11-iodo-methyl indole [3,2-b]quinoline was mixed with 60 ml of ethylene glycol diethyl ether, 0.15 mol of 4-(2-aminoethyl) piperazine was added into the above solution and stirred at 120° C. for 0.5 h, the precipitated solids were cooled, the reddish-brown solids were collected by filtration, and yellow solids were obtained by recrystallization with the mixture of ethyl ether and ethanol, the yield was 80%.

The results of structural characterization were as follows:

¹HNMR (400 Hz, DMSO-d6) δ8.60 (d, J=8.4 Hz, 1H), 8.44 (s, 1H), 8.27 (d, J=8.8 Hz, 1H), 7.92 (dd, J=8.4, 7.2 Hz, 1H), 7.72 (d, J=8.8 Hz, 1H), 7.80 (t, J=7.6 Hz, 1H), 7.48 (d, J=8.4 Hz, 1H,), 4.55 (s, 3H), 2.85 (m, 8H), 2.65 (m, 4H).

### Test Example 1

Determination of anti-tobacco Mosaic virus (TMV) activity by the following procedure:

### 1. Virus Purification and Concentration Determination:

The virus crude extract was centrifuged with polyethylene glycol at twice and refrigerated at 4° C. for use. The absorbance of 260 nm wavelength was determined by ultraviolet spectrophotometer, and the virus concentration was calculated according to the formula.

Virus concentration (mg/ml)=(A₂₆₀×dilution ratio)/E^{0.1%}_{1 cm}^{260 nm}.

Wherein E represents the extinction coefficient, that is, the light absorption (optical density) value of a suspension with a concentration of 0.1% (1 mg/ml) at a wavelength of 260 nm and an optical path of 1 cm.

The E0.1% 1 cm260 nm of TMV is 3.1.

### 2. Preparation of Cryptolepine Salt Derivative Solution:

After weighing, cryptolepine salt derivative, ningnanmycin and the original drug of ribavirin were dissolved in DMF to obtain 1×10⁵ μg/mL mother solution, and then diluted to the required concentration with 1%. Tween 80 aqueous solution.

### 3. Live Protection Function:

Select the 3-5 leaf stage of tobacco Xanthi nc with uniform growth, spray the whole plant, repeat each treatment 3 times, and set 1%. Tween80 aqueous solution as control. 24 h later, the leaf surface was coated with emery (500 mesh), the disease venom was dipped with a brush, and the whole leaf surface was gently rubbed twice along the direction of the branch pulse by using the brush, the leaf was supported by palm of hand, the virus concentration was 10 μg/mL, and the leaf was washed by water after inoculation. After 3 days, the number of disease spots was recorded and the prevention and control effect was calculated.

### 4. Live Treatment Effect:

Select the 3-5 leaf stage of tobacco Xanthi nc with uniform growth, use a brush to vaccinate the virus in the whole leaf, the virus concentration is 10 μg/mL, and the leaf was washed with running water after inoculation. After the leaves were dried, the whole plant was sprayed with test sample, repeated 3 times every treatment, and 1%. Tween 80 aqueous solution was set as control. After 3 days, the number of disease spots was recorded and the prevention and control effect was calculated.

### 5. Living Passivation Activity Test:

Select the 3-5 leaf stage of tobacco Xanthi nc with uniform growth, mix the test sample agent with the same volume of virus juice to passivate for 30 min, friction inoculation, virus concentration 20 μg/mL, the leaf was washed with running water after inoculation, repeat 3 times, set 1%. Tween 80 aqueous solution as control. 3 days later, the number of disease spots was calculated.

Relative inhibition rate (%)=[(number of dead spots of control group-number of dead spots of test sample group)/number of dead spots of control group]×100%.

Anti-tobacco Mosaic virus passivation activity test of all compounds was at a treatment dose of 500 μg/mL. The positive control was the commercial anti-plant virus agent viriazole.

The test results of the anti-TMV activity of the cryptolepine salt derivatives with the structures shown in formulas (I-1) to (I-22) and (I-44) to (I-68), Ningnanmycin and ribazole were shown in Table 4 below.

TABLE 4

Relative inhibition rate (%)

| | | |
|---|---|---|
| | | Living |
| Live | Live | passivation |
| protection | treatment | activity |
| function | effect | test |

| | |
|---|---|
| Sample | Treatment dose (mg/L) |

| Number | 500 | 500 | 500 |
|---|---|---|---|
| Example 1 | 18.9 ± 2.6 | 20.3 ± 1.1 | 18.5 ± 0.6 |
| Example 2 | 26.5 ± 3.0 | 20.5 ± 0.9 | 25.3 ± 2.3 |
| Example 3 | 18.1 ± 3.1 | 19.1 ± 0.1 | 20.3 ± 0.2 |
| Example 4 | 21.6 ± 3.4 | 18.6 ± 1.2 | 14.1 ± 1.3 |
| Example 5 | 46.7 ± 4.1 | 35.8 ± 4.8 | 39.3 ± 2.5 |
| Example 6 | 48.4 ± 4.7 | 45.2 ± 3.6 | 41.0 ± 2.2 |
| Example 7 | 44.5 ± 0.4 | 43.0 ± 3.5 | 47.7 ± 3.1 |
| Example 8 | 18.8 ± 4.2 | 23.1 ± 0.5 | 18.6 ± 1.2 |
| Example 9 | 23.8 ± 3.5 | 19.1 ± 0.1 | 23.1 ± 0.9 |
| Example 10 | 17.0 ± 1.0 | 10.2 ± 0.7 | 19.1 ± 0.2 |
| Example 11 | 27.6 ± 1.4 | 30.1 ± 1.1 | 22.3 ± 1.0 |
| Example 12 | 23.4 ± 3.3 | 19.1 ± 1.0 | 20.3 ± 2.1 |
| Example 13 | 50.8 ± 1.0 | 52.0 ± 2.4 | 46.1 ± 3.9 |
| Example 14 | 15.3 ± 1.2 | 18.9 ± 1.2 | 20.1 ± 1.5 |
| Example 15 | 15.6 ± 0.4 | 22.1 ± 0.2 | 20.0 ± 1.0 |
| Example 16 | 28.1 ± 4.5 | 30.1 ± 1.1 | 18.9 ± 0.8 |
| Example 17 | 30.2 ± 1.7 | 23.5 ± 2.1 | 33.1 ± 1.1 |
| Example 18 | 45.6 ± 0.9 | 38.7 ± 2.4 | 40.8 ± 2.9 |
| Example 19 | 47.0 ± 2.8 | 42.4 ± 4.9 | 44.2 ± 3.4 |
| Example 20 | 51.3 ± 3.5 | 48.5 ± 3.0 | 53.0 ± 2.1 |
| Example 21 | 41.4 ± 3.9 | 32.3 ± 1.8 | 35.9 ± 4.0 |
| Example 22 | 14.0 ± 1.1 | 18.2 ± 1.1 | 15.5 ± 1.1 |
| Example 23 | 33.0 ± 2.9 | 30.1 ± 1.0 | 35.2 ± 1.1 |
| Example 24 | 36.9 ± 3.7 | 30.3 ± 0.8 | 35.2 ± 1.0 |
| Example 25 | 27.3 ± 3.0 | 22.1 ± 1.1 | 35.1 ± 0.1 |
| Example 26 | 43.1 ± 3.4 | 45.4 ± 1.9 | 37.0 ± 3.0 |
| Example 27 | 52.1 ± 2.8 | 46.3 ± 4.2 | 47.9 ± 1.5 |
| Example 28 | 20.5 ± 1.7 | 19.6 ± 0.2 | 25.3 ± 1.2 |
| Example 29 | 38.6 ± 4.6 | 36.5 ± 2.3 | 30.5 ± 0.9 |
| Example 30 | 20.9 ± 2.9 | 21.5 ± 1.1 | 30.0 ± 0.8 |
| Example 31 | 29.5 ± 1.3 | 28.9 ± 1.1 | 30.5 ± 0.2 |
| Example 32 | 25.1 ± 4.3 | 30.1 ± 0.4 | 28.7 ± 2.1 |
| Example 33 | 57.9 ± 4.6 | 51.7 ± 3.6 | 49.8 ± 3.1 |
| Example 34 | 50.3 ± 2.1 | 43.9 ± 2.0 | 41.2 ± 4.6 |
| Example 35 | 41.0 ± 2.4 | 48.0 ± 1.5 | 44.3 ± 2.9 |
| Example 36 | 29.8 ± 2.1 | 30.1 ± 0.2 | 28.7 ± 1.1 |
| Example 37 | 31.6 ± 3.2 | 20.7 ± 1.2 | 33.6 ± 0.8 |
| Example 38 | 35.0 ± 4.1 | 40.2 ± 1.0 | 30.7 ± 1.2 |
| Example 39 | 25.4 ± 3.8 | 30.1 ± 0.2 | 28.9 ± 2.1 |
| Example 40 | 45.8 ± 3.8 | 39.5 ± 3.1 | 38.6 ± 2.5 |
| Example 41 | 40.8 ± 4.7 | 34.1 ± 2.3 | 30.6 ± 4.0 |
| Example 42 | 28.1 ± 3.0 | 30.2 ± 2.1 | 25.1 ± 0.2 |
| Example 43 | 26.1 ± 1.0 | 28.3 ± 0.2 | 30.0 ± 2.1 |
| Example 44 | 27.3 ± 4.4 | 33.1 ± 0.3 | 35.3 ± 2.1 |
| Embodiment 45 | 30.7 ± 2.6 | 28.1 ± 0.3 | 25.6 ± 1.1 |
| Example 46 | 44.7 ± 4.0 | 40.1 ± 0.9 | 46.3 ± 3.5 |
| Example 47 | 36.4 ± 2.8 | 33.3 ± 2.0 | 35.7 ± 0.3 |
| Reference Example 1 | 15.1 ± 2.0 | 10.5 ± 3.0 | 10.2 ± 1.8 |
| Reference Example 2 | 15.2 ± 1.1 | 18.1 ± 0.2 | 15.3 ± 2.1 |
| Ningnanmycin | 58.3 ± 4.1 | 56.9 ± 2.8 | 59.0 ± 2.2 |
| ribavirin | 39.5 ± 2.0 | 36.1 ± 3.7 | 38.0 ± 1.3 |

As can be seen from the data in Table 4, at a dose of 500 μg/mL, the vast majority of the examples showed better anti-TMV activity than reference example 1 and 2. Among them, the antiTMV activity of the cryptolepine salt derivative with the structures shown in formula (I-5), (I-6), (I-13), (I-20), (I-48), (I-54) and (I-55) was comparable to that of commercial Ningnanmycin. And formula (I-5), (I-6), (I-7), (I-13), (I-18), (I-19), (I-20), (I-21), (I-47), (I-48), (I-54), (I-55), (I-56), (I-61), (I-62) and (I-67) showed better TMV activity than the ribavirin.

### Test Example 2

Bactericidal activity test, the determination procedure is as follows:

Test of resistance to rice sheath blight: Select uniformly growing rice seedlings and spray them on the leaves at the set concentration, set a water spraying group as blank control, every treatment was repeated twice. After 24 h, the rice seedlings were inoculated with rice sheath blight strains and placed at room temperature (25±4° C.) for normal management after inoculation. The prevention and control effect was investigated by visual inspection 6 days after inoculation.

The results of bactericidal activity test of cryptolepine salt derivative with structures shown in formulas (I-1) to (I-22) and (I-44) to (I-68) and thifluzamide were shown in Table 5 below:

TABLE 5

| | Resistance to rice |
|---|---|
| Samples | sheath blight (%)/400 mg/L |
| Example 1 | 95 ± 5 |
| Example 2 | 40 ± 0 |
| Example 3 | 35 ± 5 |
| Example 4 | 60 ± 0 |
| Example 5 | 55 ± 5 |
| Example 6 | 40 ± 0 |
| Example 7 | 40 ± 0 |
| Example 8 | 30 ± 0 |
| Example 9 | 70 ± 0 |
| Example 10 | 30 ± 0 |
| Example 11 | 40 ± 0 |
| Example 12 | 40 ± 0 |
| Example 13 | 35 ± 5 |
| Example 14 | 60 ± 0 |
| Example 15 | 40 ± 0 |
| Example 16 | 95 ± 5 |
| Example 17 | 40 ± 0 |
| Example 18 | 45 ± 5 |
| Example 19 | 20 ± 0 |
| Example 20 | 55 ± 5 |
| Example 21 | 40 ± 0 |
| Example 22 | 45 ± 5 |
| Example 23 | 30 ± 0 |
| Example 24 | 30 ± 0 |
| Example 25 | 20 ± 0 |
| Example 26 | 35 ± 5 |
| Example 27 | 30 ± 0 |
| Example 28 | 60 ± 0 |
| Example 29 | 30 ± 0 |
| Example 30 | 35 ± 5 |
| Example 31 | 25 ± 5 |
| Example 32 | 30 ± 0 |
| Example 33 | 40 ± 0 |
| Examples 34 | 20 ± 0 |
| Example 35 | 25 ± 5 |
| Example 36 | 35 ± 5 |
| Example 37 | 40 ± 0 |
| Example 38 | 30 ± 0 |
| Example 39 | 25 ± 5 |
| Example 40 | 20 ± 0 |
| Example 41 | 40 ± 0 |
| Example 42 | 25 ± 5 |
| Example 43 | 30 ± 0 |
| Example 44 | 55 ± 5 |
| Example 45 | 30 ± 0 |
| Example 46 | 30 ± 0 |
| Example 47 | 45 ± 5 |
| Reference Example 1 | 0 |
| Reference Example 2 | 0 |
| Thifluzamide | 100 |

From the data in Table 5, it can be seen that in the live pot test, the cryptolepine salt derivative showed certain bactericidal activity against rice sheath blight, but reference examples 1 and 2 had no activity. Among them, the cryptolepine salt derivative shown in formula (I-1) and formula (I-16) showed more than 95% bactericidal activity against rice sheath blight. And the cryptolepine salt derivative shown in formula (I-4), (I-5), (I-9), (I-14), (I-18), (I-20), (I-22), (I-49), (I-65) and (I-68) showed more than 50% bactericidal activity against rice sheath blight.

The product obtained by examples 1-47 was characterized to prove the structure correct, and the results are shown in Table 6.

TABLE 6

| Example | | |
|---|---|---|
| serial | Corresponding | |
| number | compound | Characterization result |
| Example 1 | formula (I-1) | ¹HNMR (400 MHz,DMSO-d6) δ 9.98 (d, J = 10.7 Hz, 1H), 8.96-8.84 (m, 3H), |
| | | 8.56-8.46 (m, 1H), 8.34-8.27 (m, 1H), 8.16-8.04 (m, 4H), 7.81-7.75 (m, 1H), 7.39 |
| | 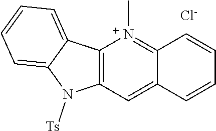 [SMILES: Cc1ccc(S(=O)(=O)n2c3ccccc3c3c2cc2ccccc2[n+]3C)cc1 ; [Cl-]] | (d, J = 8.4 Hz, 2H), 5.03 (s, 3H), 2.27 (s, 3H). 13CNMR (100 MHz, DMSO-d6) δ 147.5, 142.1, 137.6, 136.0, 135.2, 133.0, 131.7, 131.6, 131.1, 129.1, 128.3, 127.7, 127.4, 126.3, 118.9, 118.2, 115.2, 41.7, 21.5. HRMS(ESI), calculated for C₂₃H₁₉N₂O₂S⁺ [M − Cl]⁺ 387.1162, found 387.1160. |
| | I-1 | |
| Example 2 | formula (I-2) | ¹HNMR (400 MHz, DMSO-d6) δ 9.98 (s, 1H), 8.84 (d, J = 8.8 Hz, 2H), 8.71 (s, |
| | | 1H), 8.46 (d, J = 8.7 Hz, 1H), 8.34-8.25 (m, 1H), 8.12-8.01 (m, 3H), 7.95 (d, |
| | 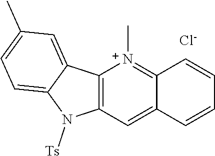 [SMILES: Cc1ccc(S(=O)(=O)n2c3ccc(C)cc3c3c2cc2ccccc2[n+]3C)cc1 ; [Cl-]] | J = 8.8 Hz, 1H), 7.37 (d, J = 8.1 Hz, 2H), 4.96 (s, 3H), 2.60 (s, 3H), 2.27 (s, 3H). ¹³CNMR (100 MHz, DMSO-d6) δ 147.4, 142.3, 140.6, 137.6, 137.2, 136.2, 135.1, 133.0, 132.0, 131.7, 131.0, 129.1, 129.0, 128.5, 127.7, 127.4, 126.0, 118.8, 118.5, 115.1,41.5,21.5,21.2. HRMS(ESI), calculated for C₂₄H₂₁N₂O₂S⁺ [M −Cl]⁺ 401.1318, found 401.1315. |
| | I-2 | |
| Example 3 | formula (I-3) | ¹HNMR (400 MHz, DMSO-d6) δ 9.94 (s, 1H), 8.91-8.82 (m, 2H), 8.42 (d, J = |
| | | 9.3 Hz, 1H), 8.34-8.27 (m, 1H), 8.17 (s, 1H), 8.10-8.05 (m, 1H), 8.00 (d, J = |
| | 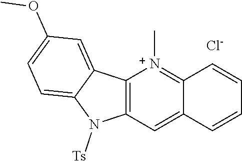 [SMILES: COc1ccc2c(c1)c1c(cc3ccccc3[n+]1C)n2S(=O)(=O)c1ccc(C)cc1 ; [Cl-]] | 8.3 Hz, 2H), 7.74-7.68 (m, 1H), 7.35 (d, J = 8.1 Hz, 2H), 5.01 (s, 3H), 4.02 (s, 3H), 2.26 (s, 3H) ¹³CNMR (100 MHz, DMSO-d6) δ 157.4, 147.4, 141.9, 137.7, 136.7, 135.3, 132.9, 132.1, 131.7, 131.0, 129.4, 129.1, 127.6, 127.4, 124.5, 119.2, 118.9, 116.4, 110.1,56.9,41.5,21.5. HRMS(ESI), calculated for C₂₄H₂₁N₂O₃S⁺ [M − Cl]⁺ 417.1267, found 417.1265. |
| | I-3 | |
| Example 4 | formula (I-4) | ¹HNMR (400 MHz, DMSO-d6) δ 10.04 (s, 1H), 8.92-8.79 (m, 3H), 8.60 (dd, |
| | | J = 9.3, 4.3 Hz, 1H), 8.38-8.28 (m, 1H), 8.15-7.99 (m, 4H), 7.39 (d, J = 8.2 Hz, |
| | 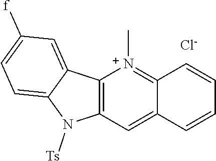 | 2H), 4.97 (s, 3H), 2.29 (s,3H). ¹³CNMR (100 MHz, DMSO-d6) δ 147.6, 141.7, 138.6, 137.7, 135.6, 132.9, 132.3, 131.7, 131.1, 129.8, 129.3, 127.7, 123.9, 123.6, 119.4, 118.9, 117.1, 114.2, 114.0,41.4,21.5. HRMS(ESI), calculated for C₂₃H₁₈FN₂O₂S⁺ [M − Cl]⁺ 405.1068, found 405.1066. |
| | I-4 | |
| Example 5 | formula (I-5) | ¹HNMR (400 MHz, DMSO-d6) δ 10.05 (s, 1H), 8.97 (s, 1H), 8.87 (d, J = 8.2 |
| | | Hz, 2H), 8.59 (d, J = 9.1 Hz, 1H), 8.36-8.29 (m, 1H), 8.21-8.04 (m, 4H), 7.40 |
| | 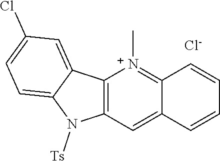 [SMILES: Cc1ccc(S(=O)(=O)n2c3ccc(Cl)cc3c3c2cc2ccccc2[n+]3C)cc1 ; [Cl-]] | (d, J = 8.1 Hz, 2H), 4.98 (s, 3H), 2.29 (s, 3H). ¹³CNMR (100 MHz, DMSO-d6) δ 147.7, 141.2, 140.6, 137.7, 135.6, 135.5, 132.9, 132.1, 131.8, 131.2, 130.8, 129.8, 129.4, 127.8, 127.2, 119.8, 118.9, 116.9, 110.0,41.6,21.5. HRMS(ESI), calculated for C₂₃H₁₈ClN₂O₂S⁺ [M − Cl]⁺ 421.0772, found 421.0771. |
| | I-5 | |
| Example 6 | formula (I-6) | ¹HNMR (400 MHz, DMSO-d6) δ 10.04 (s, 1H), 9.06 (s, 1H), 8.87 (d, J = 8.2 |
| | | Hz, 2H), 8.52 (d, J = 9.0 Hz, 1H), 8.35-8.29 (m, 1H), 8.27 (d, J = 9.1 Hz, 1H), |
| | 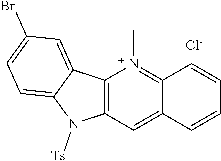 [SMILES: Cc1ccc(S(=O)(=O)n2c3ccc(Br)cc3c3c2cc2ccccc2[n+]3C)cc1 ; [Cl-]] | 8.14-8.07 (m, 3H), 7.39 (d, J = 8.3 Hz, 2H), 4.98 (s, 3H), 2.29 (s, 3H). ¹³CNMR (100 MHz, DMSO-d6) δ 147.7, 141.1, 141.0, 138.3, 137.7, 135.6, 132.9, 131.9, 131.7, 131.2, 130.0, 129.7, 129.4, 127.8, 120.2, 118.9, 118.8, 117.1, 41.7, 21.5. HRMS(ESI), calculated for C₂₃H₁₈BrN₂O₂S⁺ [M − Cl]⁺ 465.0267, found 465.0269. |
| | I-6 | |
| Example 7 | formula (1-7) | ¹HNMR (400 MHz, CDCl3) δ 8.79 (s, 1H), 8.57-8.46 (m, 2H), 8.07 (t, J = 7.8 |
| | | Hz, 1H), 7.96 (d, J = 8.0 Hz, 2H), 7.83 (d, J = 8.1 Hz, 1H), 7.65 (t, J = 7.5 Hz, |
| | 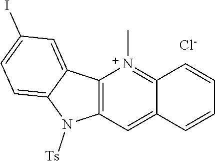 [SMILES: Cc1ccc(S(=O)(=O)n2c3ccc(I)cc3c3c2cc2ccccc2[n+]3C)cc1 ; [Cl-]] | 1H), 7.54 (d, J = 8.7 Hz, 1H), 7.33 (d, J = 8.8 Hz, 1H), 7.30-7.18 (m, 3H), 4.96 (s, 3H), 2.38 (s, 3H). 13CNMR(100MHz,CDCl3)0144.4, 142.9, 141.6, 140.0, 135.2, 134.7, 13 3.4, 132.7, 132.5, 129.7, 128.9, 127.3, 126.2, 126. 1, 125.8, 117.5, 115.5, 114.4, 40.5,21.4. HRMS(ESI), calculated for C₂₃H₁₈IN₂O₂S⁺ [M − Cl]⁺ 513.0128, found 513.0129. |
| | I-7 | |
| Example 8 | formula (I-8) | ¹HNMR (400 MHz, CDCl3) δ 8.07 (d, J = 8.6 Hz, 1H), 7.88 (s, 1H), 7.77 (d, J = |
| | | 8.3 Hz, 2H), 7.69 (d, J = 3.7 Hz, 1H), 7.59-7.53 (m, 1H), 7.28 (s, 1H), 6.72 (d, |
| | 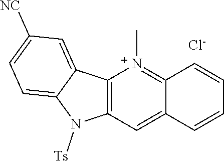 [SMILES: Cc1ccc(S(=O)(=O)n2c3ccc(C#N)cc3c3c2cc2ccccc2[n+]3C)cc1 ; [Cl-]] | J = 3.7 Hz, 1H), 2.37 (s, 3H). ¹³CNMR (100 MHz, CDCl3) δ 145.8, 136.4, 134.8, 130.7, 130.2, 128.4, 127.6, 126.9, 126.4, 119.3, 114.3, 108.5, 106.9,21.7. HRMS(ESI), calculated for C₂₄H₁₈N₃O₂S⁺ [M − Cl]⁺ 412.1114, found 412.1117. |
| | I-8 | |
| Example 9 | formula (I-9) | ¹HNMR (400 MHz,DMSO-d6) δ 9.88 (s, 1H), 8.86 (d, J = 8.4 Hz, 1H), 8.72 (d, |
| | | J = 7.9 Hz, 1H), 8.58 (d, J = 9.2 Hz, 2H), 8.15 (d, J = 9.3 Hz, 1H), 8.13-8.09 (m, |
| | 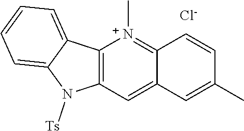 [SMILES: Cc1ccc(S(=O)(=O)n2c3ccccc3c3c2cc2cc(C)ccc2[n+]3C)cc1 ; [Cl-]] | 1H), 8.07 (d, J = 8.0 Hz, 2H), 7.81-7.74 (m, 1H), 7.38 (d, J = 8.1 Hz, 2H), 4.94 (s, 3H), 2.68 (s, 3H), 2.28 (s, 3H). ¹³CNMR (100 MHz, DMSO-d6) δ 147.5, 142.1, 141.6, 139.4, 137.2, 136.2, 135.7, 133.1, 131.9, 131.7, 131.1, 130.1, 130.0, 128.3, 128.1, 127.7, 127.7, 126.3, 118.5, 118.5, 115.4,41.4,21.5,21.3. HRMS(ESI), calculated for C₂₄H₂₁N₂O₂S⁺ [M − Cl]⁺ 401.1318, found 401.1318. |
| | I-9 | |
| Example 10 | formula (1-10) | ¹HNMR (400 MHz, DMSO-d6) δ 9.86 (s, 1H), 8.84 (d, J = 8.3 Hz, 1H), 8.77 (d, |
| | | J = 9.8 Hz, 1H), 8.54 (d, J = 8.6 Hz, 1H), 8.31 (d, J = 2.8 Hz, 1H), 8.06 (d, J = |
| | 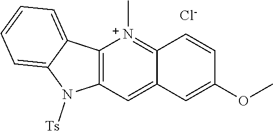 [SMILES: COc1ccc2c(c1)cc1c(c3ccccc3n1S(=O)(=O)c1ccc(C)cc1)[n+]2C ; [Cl-]] | 8.4 Hz, 3H), 7.93-7.88 (m, 1H), 7.78-7.72 (m, 1H), 7.39 (d, J = 8.3 Hz, 2H), 4.95 (s, 3H), 4.07 (s, 3H), 2.29 (s, 3H). ¹³CNMR (100 MHz, DMSO-d6) δ 159.0, 147.5, 141.7, 140.0, 135.3, 133.4, 133.1, 132.2, 131.1, 129.6, 127.8, 127.6, 127.4, 127.2, 126.2, 120.5, 118.5, 115.3, 109.2, 56.8, 41.7, 21.5. HRMS(ESI), calculated for C₂₄H₂₁N₂O₃S⁺ [M − Cl]⁺ 417.1267, found 417.1268. |
| | I-10 | |
| Example 11 | formula (I-11) | ¹HNMR (400 MHz, DMSO-d6) δ 10.00 (s, 1H), 8.95 (d, J = 22.6 Hz, 2H), 8.75 |
| | | (s, 1H), 8.57 (d, J = 7.1 Hz, 1H), 8.27 (s, 1H), 8. 10 (s,3 H), 7.79 (s, 1H), 7.41 |
| | 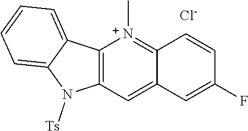 [SMILES: Cc1ccc(S(=O)(=O)n2c3ccccc3c3c2cc2cc(F)ccc2[n+]3C)cc1 ; [Cl-]] | (s, 2H), 5.00 (s, 3H), 2.29 (s, 3H). ¹³CNMR (100 MHz, DMSO-d6) δ 147.6, 142.4, 142.3, 136.2, 134.8, 133.0, 132.6, 131.1, 128.4, 128.2, 127.8, 126.4, 124.8, 124.5, 122.4, 118.2, 115.4, 114.9, 114.7, 42.1, 21.5. ¹⁹FNMR (376 MHz, DMSO) δ −110.17-−110.42(m). HRMS(ESI), calculated for C₂₃H₁₈FN₂O₂S⁺ [M − Cl]⁺ 405.1068, found 405.1068. |
| | I-11 | |
| Example 12 | formula (I-12) | ¹HNMR (400 MHz, DMSO-d6) δ 9.98 (s, 1H), 9.03 (s, 1H), 8.95-8.86 (m, 2H), |
| | | 8.58 (d, J = 8.6 Hz, 1H), 8.32 (d, J = 9.6 Hz, 1H), 8.15 (t, J = 8.0 Hz, 1H), 8.10 |
| | 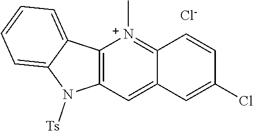 [SMILES: Cc1ccc(S(=O)(=O)n2c3ccccc3c3c2cc2cc(Cl)ccc2[n+]3C)cc1 ; [Cl-]] | (d, J = 8.0 Hz, 2H), 7.79 (t, J = 7.8 Hz, 1H), 7.40 (d, J = 8.1 Hz, 2H), 4.98 (s, 3H), 2.29 (s, 3H). ¹³CNMR (100 MHz, DMSO-d6) δ 147.6, 142.8, 142.5, 136.4, 136.2, 134.9, 133.6, 133.0, 132.6, 131.1, 129.9, 128.5, 128.3, 128.0, 127.8, 126.4, 121.3, 118.2, 115.4, 100.0, 41.9, 21.5. HRMS(ESI), calculated for C₂₃H₁₈ClN₂O₂S⁺ [M − Cl]⁺ 421.0772, found 421.0773. |
| | I-12 | |
| Example 13 | formula (I-13) | ¹HNMR (400 MHz, DMSO-d6) δ 9.97 (s, 1H), 9.18 (d, J = 2.1 Hz, 1H), 8.91 (d, |
| | | J = 8.3 Hz, 1H), 8.83 (d, J = 9.6 Hz, 1H), 8.58 (d, J = 8.6 Hz, 1H), 8.41 (d, J = |
| | 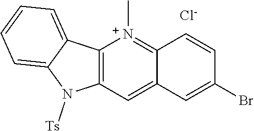 [SMILES: Cc1ccc(S(=O)(=O)n2c3ccccc3c3c2cc2cc(Br)ccc2[n+]3C)cc1 ; [Cl-]] | 9.5 Hz, 1H), 8.17-8.12 (m, 1H), 8.10 (d, J = 8.4 Hz, 2H), 7.82-7.74 (m, 1H), 7.39 (d, J = 8.3 Hz, 2H), 4.97 (s, 3H), 2.29 (s, 3H). ¹³CNMR (100 MHz, DMSO-d6) δ 147.6, 142.7, 142.5, 137.4, 136.5, 136.4, 133.2, 133.0, 132.4, 131. 1, 128.7, 128.5, 127.9, 127.8, 126.4, 122.2, 121.2, 118.2, 115.4, 41.9, 21.5. HRMS(ESI), calculated for C₂₃H₁₈BrN₂O₂S⁺ [M − Cl]⁺ 465.0267, found 465.0268. |
| | I-13 | |
| Example 14 | formula (I-14) | ¹HNMR (400 MHz, DMSO-d6) δ 10.23 (s, 1H), 9.45 (s, 1H), 9.09 (d, J = 9.4 |
| | | Hz, 1H), 8.97 (d, J = 8.3 Hz, 1H), 8.63 (d, J = 8.6 Hz, 1H), 8.59-8.52 (m, 1H), |
| | 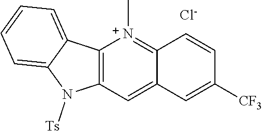 [SMILES: Cc1ccc(S(=O)(=O)n2c3ccccc3c3c2cc2cc(C(F)(F)F)ccc2[n+]3C)cc1 ; [Cl-]] | 8.19 (t, J = 8.0 Hz, 1H), 8. 14 (d, J = 8.5 Hz, 2H), 7.82 (t, J = 7.7 Hz, 1H), 7.41 (d, J = 8.2 Hz, 2H), 5.03 (s, 3H), 2.30 (s, 3H). ¹³CNMR (100 MHz, DMSO-d6) δ 147.7, 144.2, 142.8, 138.8, 136.9, 133.0, 132.7, 131.2, 129.9, 128.8, 127.8, 126.7, 126.5, 121.0, 118.1, 115.4, 42.0, 21.5. ¹⁹FNMR (376 MHz, DMSO) δ-61.03 (s). HRMS(ESI), calculated for C₂₄H₁₈F₃N₂O₂S⁺ [M − Cl]⁺ 455.1036, found 455.1039. |
| | I-14 | |
| Example 15 | formula (I-15) | ¹HNMR (400 MHz, DMSO-d6) δ 10.21 (s, 1H), 9.54 (s, 1H), 8.94 (t, J = 7.8 Hz, |
| | | 2H), 8.63 (d, J = 8.4 Hz, 2H), 8.18 (t, J = 8.0 Hz, 3H), 7.81 (t, J = 7.6 Hz, 1H), |
| | 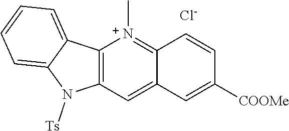 [SMILES: COC(=O)c1ccc2c(c1)cc1c(c3ccccc3n1S(=O)(=O)c1ccc(C)cc1)[n+]2C ; [Cl-]] | 7.40 (d, J = 8.2 Hz, 2H), 4.99 (s, 3H), 4.04 (s, 3H), 2.29 (s, 3H). ¹³CNMR (100 MHz, DMSO-d6) δ 165.3, 147.7, 143.7, 142.8, 139.2, 136.7, 133.9, 133.3, 133.1, 132.3, 131.1, 130.2, 129.4, 128.6, 127.8, 127.0, 126.4, 119.8, 118.1, 115.4, 53.5, 41.9, 21.5. HRMS(ESI), calculated for C₂₅H₂₁N₂O₄S⁺ [M − Cl]⁺ 445.1217, found 445.1216. |
| | I-15 | |
| Example 16 | formula (I-16) | ¹HNMR (400 MHz, DMSO-d6) δ 9.95 (s, 1H), 8.89 (d, J = 8.2 Hz, 1H), 8.72 (d, |
| | | J = 8.4 Hz, 1H), 8.68 (s, 1H), 8.57 (d, J = 8.6 Hz, 1H), 8.12 (d, J = 7.8 Hz, 1H), |
| | 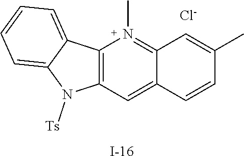 | 8.07 (d, J = 8.4 Hz, 2H), 7.93 (d, J = 8.4 Hz, 1H), 7.77 (t, J = 7.7 Hz, 1H), 7.38 (d, J = 8.2 Hz, 2H), 4.94 (s, 3H), 2.76 (s, 3H), 2.28 (s, 3H). ¹³CNMR (100 MHz, DMSO-d6) δ 147.5, 147.1, 142.0, 141.7, 137.9, 135.6, 133.1, 131.2, 131.1, 129.1, 128.1, 127.7, 126.2, 125.9, 118.5, 117.7, 115.4, 41.3, 22.8, 21.5. HRMS(ESI), calculated for C₂₄H₂₁N₂O₂S⁺ [M − Cl]⁺ 401.1318, found 401.1318. |
| Example 17 | formula (I-17) | ¹HNMR (400 MHz, DMSO-d6) δ 10.05 (s, 1H), 9.02 (s, 1H), 8.96-8.85 (m, 2 |
| | | H), 8.59 (d, J = 8.6 Hz, 1H), 8.20-8.13 (m, 2H), 8.10 (d, J = 8.4 Hz, 2H), 7.79 |
| | 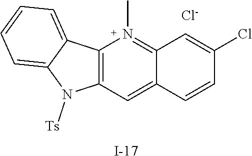 | (t, J = 7.8 Hz, 1H), 7.40 (d, J = 8.3 Hz, 2H), 4.95 (s, 3H), 2.29 (s, 3H). ¹³CNMR (100 MHz, DMSO-d6) δ 147.1, 142.4, 142.0, 139.8, 137.6, 135.9, 132.9, 132.6, 131.5, 130.6, 129.3, 128.6, 127.9, 127.3, 125.9, 125.7, 117.9, 117.7, 114.9, 41.3, 21.0. HRMS(ESI), calculated for C₂₃H₁₈ClN₂O₂S⁺ [M −Cl]⁺ 421.0772, found 421.0773. |
| Example 18 | formula (I-18) | ¹HNMR (400 MHz, DMSO-d6) δ 10.06 (s, 1H), 8.86 (t, J = 9.8 Hz, 2H), 8.79 (d) |
| | | J = 8.1 Hz, 1H), 8.59 (d, J = 8.3 Hz, 1H), 8.32 (t, J = 7.7 Hz, 1H), 8.19-8.05 (m, |
| | 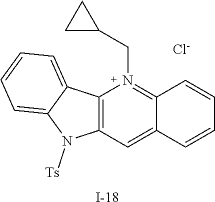 | 4H), 7.84 (t, J = 7.3 Hz, 1H), 7.41 (d, J = 7.3 Hz, 2H), 5.52 (d, J = 4.9 Hz, 2H), 2.30 (s, 3H), 1.51 (s, 1H), 0.82 (s, 2H), 0.66 (d, J = 7.2 Hz, 2H). ¹³CNMR (100 MHz, DMSO-d6) δ 147.5, 142.5, 141.5, 137.2, 136.0, 135.3, 133.3, 132.3, 131.9, 131.1, 129.7, 129.2, 127.8, 127.4, 126.6, 119.0, 117.5, 115.6, 54.7, 21.6, 10.6, 4.6. HRMS(ESI), calculated for C₂₆H₂₃N₂O₂S⁺ [M − Cl]⁺ 427.1475 , found 427.1474. |
| Example 19 | formula (1-19) | ¹HNMR (400 MHz, DMSO-d6) δ 10.07 (s, 1H), 8.96 (d, J = 9.2 Hz, 1H), 8.87 |
| | | (d, J = 7.9 Hz, 1H), 8.64-8.56 (m, 2H), 8.32-8.25 (m, 1H), 8.17-8.06 (m, 4H), |
| | 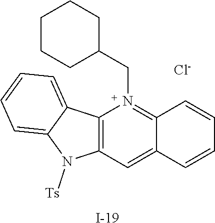 | 7.83 (t, J = 7.8 Hz, 1H), 7.40 (d, J = 8.3 Hz, 2H), 5.44 (d, J = 92.5 Hz, 2H), 2.89 (s, 1H), 2.72 (s, 1H), 2.51 (s, 1H), 2.30 (s, 3H), 2.09 (s, 1H), 1.83 (s, 1H), 1.62-1.53 (m, 2H), 1.43-1.34 (m, 2H), 1.19-1.04 (m, 2H). ¹³CNMR (100 MHz, DMSO-d6) δ 147.5, 142.4, 141.7, 137.7, 135.8, 135.1, 133.2, 132.4, 131.8, 131.1, 129.8, 129.2, 127.8, 127.7, 127.2, 126.6, 119.4, 117.7, 115.6, 56.2, 38.0, 36.3, 31.2, 26.0, 21.5. HRMS(ESI), calculated for C₂₉H₂₉N₂O₂S⁺ [M − Cl]⁺ 469.1944, found 469.1946. |
| Example 20 | formula (I-20) | ¹HNMR (400 MHz, DMSO-d6) δ 10.12 (s, 1H), 9.09 (d, J = 7.6 Hz, 1H),9.01- |
| | | 8.83 (m, 2H), 8.56 (d, J = 7.9 Hz, 1H), 8.26 (s, 1H), 8.11-7.94 (m, 4H), 7.77 |
| | 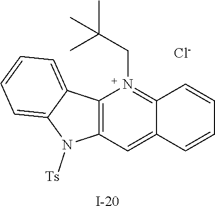 | (s, 1H), 7.35 (d, J = 7.0 Hz, 2H), 5.77 (s, 1H), 5.55 (d, J = 14.7 Hz, 1H), 2.27 (s, 3H), 0.85 (s, 9H). ¹³CNMR (100 MHz, DMSO-d6) δ 147.5, 142.9, 142.4, 138.7, 135.9, 134.5, 132.9, 132.6, 131.7, 131.0, 129.3, 128.2, 127.9, 127.7, 126.2, 120.7, 118.6, 115.9, 59.0, 37.4, 28.6, 21.5. HRMS(ESI), calculated for C₂₇H₂₇N₂O₂S⁺ [M − Cl]⁺ 443.1788, found 443.1790. |
| Example 21 | formula (I-21) | ¹HNMR (400 MHz, CDCl3) δ 9.87 (s, 1H), 8.61 (d, J = 9.1 Hz, 1H), 8.55 |
| | | (d, J = 8.6 Hz, 1H), 8.50 (d, J = 8.1 Hz, 1H), 8.29 (d, J = 8.3 Hz, 1H), 8.17 |
| | 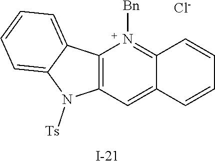 | (t, J = 7.5 Hz, 1H), 7.99-7.92 (m, 2H), 7.83 (d, J = 8.4 Hz, 2H), 7.57 (t, J = 7.8 Hz, 1H), 7.33 (d, J = 5.2 Hz, 3H), 7.31-7.26 (m, 6H), 2.33 (s, 3H). ¹³CNMR (100 MHz, CDCl3) δ 147.5, 143.3, 141.9, 137.7, 136.0, 133.0, 132.1, 131.9, 130.8, 130.7, 130.0, 129.7, 129.5, 128.8, 127.2, 127.0, 126.7, 126.6, 125.9, 119.3, 116.9, 115.7, 57.5, 21.7. HRMS(ESI), calculated for C₂₉H₂₃N₂O₂S⁺ [M − Cl]⁺ 463.1475, found 463.1476. |
| Example 22 | formula (I-22) | ¹HNMR (400 MHz, DMSO-d6) δ 10.03 (s, 1H), 8.92 (d, J = 8.2 Hz, 1H), 8.85 |
| | | (d, J = 8.6 Hz, 2H), 8.58 (d, J = 8.5 Hz, 1H), 8.31 (t, J = 7.6 Hz, 1H), 8.21 (d, |
| | 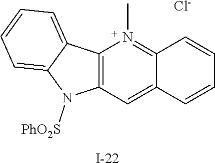 | J = 7.7 Hz, 2H), 8.16-8.07 (m, 2H), 7.79 (t, J = 7.7 Hz, 1H), 7.74 (t, J = 7.4 Hz, 1H), 7.60 (t, J = 7.7 Hz, 2H), 4.98 (s, 3H). ¹³CNMR (100 MHz, DMSO-d6) δ 142.4, 142.2, 137.6, 136.4, 136.0, 135.2, 131.8, 131.7, 130.7, 129.2, 129.1, 128.4, 127.7, 127.5, 126.4, 118.8, 118.4, 115.4, 110.0, 41.5. HRMS(ESI), calculated for C₂₂H₁₇N₂O₂S⁺ [M − Cl]⁺ 373.1005, found 373.1004. |
| Example 23 | formula (I-44) | ¹HNMR (400 MHz, DMSO-d6) δ 11.64 (s, 1H), 8.69 (d, J = 8.5 Hz, 2H), 8.51 |
| | | (d, J = 8.4 Hz, 1H), 8.30 (d, J = 8.9 Hz, 1H), 8.04-7.96 (m, 1H), 7.84 (d, J = |
| | 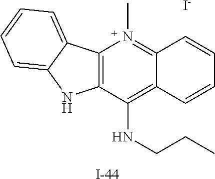 | 8.4 Hz, 1H), 7.75-7.65 (m, 2H), 7.41-7.31 (m, 1H), 4.55 (s, 3H), 4.04 (t, J = 6.4 Hz, 2H), 1.94-1.79 (m, 2H), 1.04 (t, J = 7.3 Hz, 3H). ¹³CNMR (100 MHz, DMSO-d6) δ 144.0, 142.9, 137.5, 135.8, 132.7, 130.9, 124.7, 124.4, 124.4, 121.2, 117.8, 116.6, 115.5, 114.8, 113.9,47.4, 38.5, 23.5, 11.7. HRMS(ESI), calculated for C₁₉H₂₀N₃⁺ [M − I]⁺ 290.1652, found 290.1651. |
| Example 24 | formula (I-45) | ¹HNMR (400 MHz, DMSO-d₆) δ 11.61 (s, 1H), 8.69 (d, J = 7.7 Hz, 2H), 8.50 |
| | | (d, J = 7.9 Hz, 1H), 8.29 (d, J = 8.5 Hz, 1H), 8.06-7.95 (m, 1H), 7.84 (d, J = |
| | 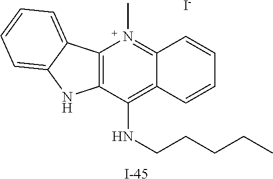 | 7.8 Hz, 1H), 7.76-7.61 (m, 2H), 7.42-7.28 (m, 1H), 4.54 (s, 3H), 4.06 (s, 2H), 1.93-1.69 (m, 2H), 1.53-1.30 (m, 4H), 0.90 (t, J = 6.4 Hz, 3H). ¹³CNMR (100 MHz, DMSO-d6) δ 143.5, 142.4, 137.0, 135.3, 132.2, 130.4, 124.2, 123.9, 120.7, 117.3, 116.1, 115.0, 114.3, 113.4, 45.4, 38.0, 29.3, 28.4, 21.9, 13.9. HRMS(ESI), calculated for C₂₁H₂₄N₃⁺ [M − I]⁺ 318.1965, found 318.1964. |
| Example 25 | formula (I-46) | ¹HNMR (400 MHz, DMSO-d₆) δ 11.62 (s, 1H), 8.69 (d, J = 7.8 Hz, 2H), 8.51 |
| | | (d, J = 8.1 Hz, 1H), 8.31 (d, J = 8.5 Hz, 1H), 8.05-7.96 (m, 1H), 7.84 (d, J = |
| | 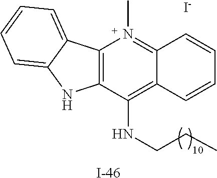 | 7.9 Hz, 1H), 7.75-7.64 (m, 2H), 7.41-7.30 (m, 1H), 4.55 (s, 3H), 4.07 (s, 2H), 1.44 (s, 2H), 1.24 (d, J = 52.3 Hz, 18H), 0.82 (t, J = 6.3 Hz, 3H). ¹³CNMR (100 MHz, DMSO-d6) δ 144.0, 143.0, 137.6, 135.8, 132.7, 130.9, 124.7, 124.4, 121.2, 117.8, 116.6, 115.6, 114.9, 113.9, 45.9, 38.5, 31.7, 30.1, 29.5, 29.4, 29.2, 26.7, 22.5, 14.4. HRMS(ESI), calculated for C₂₈H₃₈N₃⁺ [M −I]⁺ 416.3060, found 416.3060. |
| Example 26 | formula (I-47) | ¹HNMR (400 MHz, MeOH-d₄) δ 8.57 (d, J = 8.6 Hz, 1H), 8.49 (d, J = 8.5 Hz, |
| | | 1H), 8.24 (d, J = 8.9 Hz, 1H), 8.04-7.97 (m, 1H), 7.83 (d, J = 8.4 Hz, 1H), |
| | 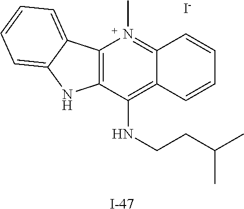 | 7.75-7.66 (m, 2H), 7.45-7.37 (m, 1H), 4.61 (s, 3H), 4.17 (t, J = 7.1 Hz, 2H), 1.89-1.78 (m, 3H), 1.03 (d, J = 6.3 Hz, 6H). ¹³CNMR (100 MHz, MeOH-d₄) δ 144.1, 143.1, 137.7, 136.1, 132.3, 130.7, 1 24.2, 123.7, 123.3, 121.0, 116.7, 115.6, 114.8, 113.1, 110.0, 44.1, 38.7, 37.3, 25.6, 21.5. HRMS(ESI), calculated for C₂₁H₂₄N₃⁺ [M − I]⁺ 318.1965, found 318.1966. |
| Example 27 | formula (I-48) | ¹HNMR (400 MHz, DMSO-d₆) δ 11.75 (s, 1H), 8.75 (d, J = 8.1 Hz, 1H), 8.59 |
| | | (d, J = 8.5 Hz, 2H), 8.38 (d, J = 8.8 Hz, 1H), 8.09-7.99 (m, 1H), 7.85 (d, J = 8.4 |
| | 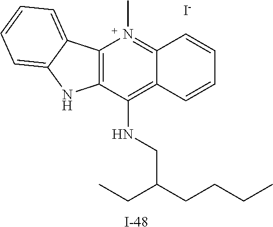 | Hz, 1H), 7.81-7.68 (m, 2H), 7.46-7.35 (m, 1H), 4.63 (s, 3H), 4.02 (s, 2H), 1.94-1.85 (m, 1H), 1.52-1.33 (m, 4H), 1.32-1.19 (m, 4H), 0.87 (t, J = 7.4 Hz, 3H), 0.82 (t, J = 7.1 Hz, 3H). ¹³CNMR (100 MHz, DMSO-d₆) δ 144.4, 143.1, 137.7, 136.3, 132.8, 131.2, 125.0, 124.6, 124.3, 121.4, 118.0, 117.3, 115.9, 115.3, 114.0, 49.6, 38.6, 30.5, 28.5, 23.9, 22.9, 14.3, 10.9. HRMS(ESI), calculated for C₂₄H₃₀N₃⁺ [M − I]⁺ 360.2434, found 360.2433. |
| Example 28 | formula (I-49) | ¹HNMR (400 MHz, DMSO-d₆) δ 11.70 (s, 1H), 8.87 (s, 1H), 8.73 (d, J = 8.5 Hz |
| | | 1H), 8.52 (d, J = 8.4 Hz, 1H), 8.32 (d, J = 8.9 Hz, 1H), 8.05-7.97 (m, 1H), 7.85 |
| | 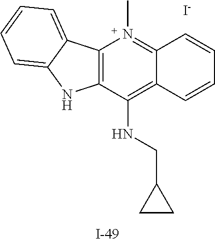 | (d, J = 83 Hz, 1H), 7.74-7.66 (m, 2H), 7.40-7.32 (m, 1H), 4.57 (s, 3H), 3.97 (d, J = 6.7 Hz, 2H, 1.38-1.26 (m, 1H), 0.63-0.54 (m, 2H), 0.52-0.42 (m, 2H). ¹³CNMR (100 MHz, DMSO-d₆) δ 143.9, 143.0, 137.6, 135.8, 132.8, 131.0, 124.7, 124.5, 124.4, 121.2, 117.8, 116.7, 115.6, 114.9, 114.0, 50.2, 38.5, 12.0, 4.3. HRMS(ESI), calculated for C₂₀H₂₀N₃⁺ [M − I]⁺ 302.1652, found 302.1650. |
| Example 29 | formula (I-50) | ¹HNMR (400 MHz, DMSO-d₆) δ 11.63 (s, 1H), 8.76 (d, J = 8.4 Hz, 1H), 8.55 |
| | | (d, J = 8.3 Hz, 1H), 8.46-8.29 (m, 2H), 8.06-7.97 (m, 1H), 7.88 (d, J = 8.3 Hz, |
| | 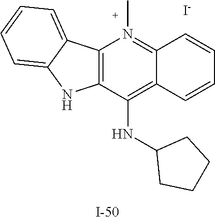 | 1H), 7.78-7.66 (m, 2H), 7.44-7.33 (m, 1H), 5.02 (s, 1H), 4.60 (s, 3H), 2.29-2.14 (m, 2H), 1.98-1.89 (m, 2H), 1.88-1.78 (m, 2H), 1.73 (d, J = 6.1 Hz, 2H). ¹³CNMR (100 MHz, DMSO-d₆) δ 143.5, 143.0, 137.6, 136.0, 132.8, 131.1, 124.9, 124.8, 124.3, 121.2, 117.8, 116.8, 115.7, 114.9, 114.0, 57.0, 38.6, 34.2, 24.5. HRMS(ESI), calculated for C₂₁H₂₂N₃⁺ [M − I]⁺ 316.1808, found 316.1808. |
| Example 30 | formula (I-51) | ¹HNMR (400 MHz, DMSO-d₆) δ 11.86 (s, 1H), 8.77 (d, J = 8.5 Hz, 2H), 8.56 |
| | | (d, J = 8.4 Hz, 1H), 8.35 (d, J = 8.9 Hz, 1H), 8.02 (t, J = 7.8 Hz, 1H), 7.88 (d, |
| | 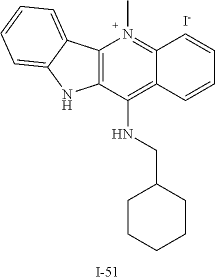 | J = 8.4 Hz, 1H), 7.79-7.67 (m, 2H), 7.39 (t, J = 7.6 Hz, 1H), 4.61 (s, 3H), 3.98 (s, 2H), 1.85 (d, J = 10.3 Hz, 3H), 1.64 (d, J = 23.4 Hz, 3H), 1.23-1.11 (m, 3H), 1.10-0.99 (m, 2H). ¹³CNMR (100 MHz, DMSO-d₆) δ 144.3, 143.0, 137.7, 136.0, 132.8, 131.0, 124.8, 124.5, 124.5, 121.3, 118.0, 117.1, 115.8, 115.1, 114.1, 54.8, 52.0, 38.5, 30.7, 26.4 ,25.8. HRMS(ESI), calculated for C₂₃H₂₆N₃⁺ [M − I]⁺ 344.2121, found 344.2122. |
| Example 31 | formula (I-52) | ¹HNMR (400 MHz, DMSO-d₆) δ 11.87 (s, 1H), 8.88 (s, 1H), 8.73 (d, J = 7.5 Hz |
| | | 1H), 8.53 (d, J = 7.4 Hz, 1H), 8.32 (d, J = 8.1 Hz, 1H), 8.05-7.97 (m, 1H), 7.87 |
| | 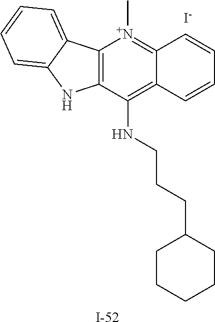 | (d, J = 7.3 Hz, 1H), 7.76-7.65 (m, 2H), 7.37 (t, J = 6.2 Hz, 1H), 4.58 (s, 3H), 4.09 (s, 2H), 1.82 (s, 2H), 1.73-1.50 (m, 6H), 1.35 (d, J = 5.1 Hz, 2H), 1.21-1.10 (m, 3H), 0.91-0.80 (m, 2H) . ¹³CNMR (100 MHz, DMSO-d₆) δ 144.1, 143.0, 137.7, 135.7, 132.7, 130.9, 124.7, 124.6, 124.3, 121.2, 117.8, 116.8, 115.6, 115.0, 114.0, 46.2, 38.5, 37.2, 34.4, 33.2, 27.6, 26.6, 26.2. HRMS(ESI), calculated for C₂₅H₃₀N₃⁺ [M − I]⁺ 372.2434, found 372.2437. |
| Example 32 | formula (I-53) | ¹HNMR (400 MHz, DMSO-d₆) δ 8.60 (d, J = 8.4 Hz, 1H), 8.42 (d, J = 8.4 Hz, 1 |
| | | H), 8.23 (d, J = 8.9 Hz, 1H), 7.95-7.89 (m, 1H), 7.72 (d, J = 8.4 Hz, 1H), 7.63- |
| | 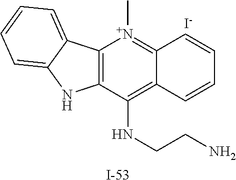 | 7.55 (m, 2H), 7.23-7.18 (m, 1H), 4.51 (s, 3H), 4.28-4.21 (m, 2H), 3.20 (s, 2H). ¹³CNMR (100 MHz, DMSO-d₆) δ 145.6, 145.0, 137.1, 135.2, 131.8, 129.5, 124.2, 124.1, 123.4, 120.8, 119.7, 117.3, 115.4, 115.4, 46.6, 40.8, 38.0. HRMS(ESI), calculated for C₁₈H₁₉N₄⁺ [M − I]⁺ 291.1604, found 291.1602. |
| Example 33 | formula (I-54) | ¹HNMR (400 MHz, DMSO-d₆) δ 8.62 (d, J = 8.2 Hz, 1H), 8.41 (d, J = 8.2 Hz, 1 |
| | | H), 8.25 (d, J = 8.8 Hz, 1H), 7.91 (t, J = 7.4 Hz, 1H), 7.67 (d, J = 8.3 Hz, 1H), |
| | 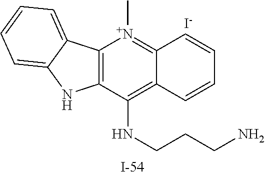 | 7.59 (t, J = 7.1 Hz, 1H), 7.50 (t, J = 7.0 Hz, 1H), 7.14-7.09 (m, 1H),4.57 (s, 3H), 4.52 (s, 2H), 3.12 (s, 1H), 2.93 (s, 2H), 2.09 (s, 2H), 1.61 (s, 2H). ¹³CNMR (100 MHz, DMSO-d₆) δ 149.5, 148.8, 145.2, 136.6, 135.4, 131.2, 128.2, 124.3, 124.0, 122.7, 118.1, 117.1, 116.9, 115.3, 114.5, 42.1, 38.6, 37.9, 30.2. HRMS(ESI), calculated for C₁₉H₂₁N₄⁺ [M − I]⁺ 305.1761, found 305.1759. |
| Example 34 | formula (I-55) | ¹HNMR (400 MHz, DMSO-d₆) δ 8.69 (d, J = 8.5 Hz, 1H), 8.51 (d, J = 8.4 Hz, 1 |
| | | H), 8.31 (d, J = 8.9 Hz, 1H), 8.01-7.95 (m, 1H), 7.81 (d, J = 8.5 Hz, 1H), 7.71- |
| | 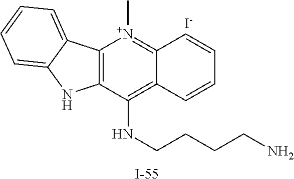 | 7.61 (m, 2H), 7.28 (t, J = 7.6 Hz, 1H), 4.59 (s, 3H), 4.23 (t, J = 6.7 Hz, 2H), 2.90 (t, J = 7.1 Hz, 2H) 1.94-1.83 (m, 2H), 1.78-1.66 (m, 2H). ¹³CNMR (100 MHz, DMSO-d₆) δ 145.6, 144.2, 137.3, 135.7, 132.2, 129.9, 124.5, 124.3, 123.7, 120.0, 119.6, 117.6, 115.3, 115.2, 115.0, 44.7, 39.1, 38.3, 27.4, 24.9. HRMS(ESI), calculated for C₂₀H₂₃N₄⁺ [M − I]⁺ 319.1917, found 319.1918. |
| Example 35 | formula (I-56) | ¹HNMR (400 MHz, DMSO-d₆) δ 8.64 (d, J = 8.3 Hz, 1H), 8.41 (d, J = 8.5 Hz, 1 |
| | | H), 8.21 (d, J = 8.9 Hz, 1H), 7.91 (t, 1H), 7.71 (d, J = 8.4 Hz, 1H), 7.63-7.53 (m, |
| | 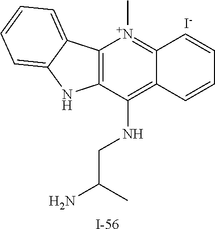 | 2H), 7.19 (t, 1H), 4.51 (s, 3H), 3.50 (t, J = 10.6, 6.6 Hz, 1H), 1.85 (s, 2H), 1.33 (d, J = 6.5 Hz, 3H). ¹³CNMR (100 MHz, DMSO-d₆) δ 170.2, 145.2, 144.8, 136.8, 134.5, 131.3, 128.8, 123.8, 123.7, 122.8, 120.7, 119.1, 116.7, 115.3, 115.0, 46.7, 37.5, 22.6, 17.2. HRMS(ESI), calculated for C₁₉H₂₁N₄⁺ [M − I]⁺ 305.1761, found 305.1760. |
| Example 36 | formula (I-57) | ¹HNMR (400 MHz, DMSO-d₆) δ 8.73 (d, J = 8.4 Hz, 1H), 8.40 (d, J = 8.4 Hz, 1 |
| | | H), 8.15 (d, J = 8.8 Hz, 1H), 7.91-7.85 (m, 1H), 7.69 (d, J = 8.4 Hz, 1H), 7.59- |
| | 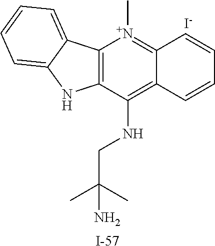 | 7.50 (m, 2H), 7.21-7.15 (m, 1H), 4.49 (s, 3H), 4. 14 (s, 2H), 1.88 (s, 1H), 1.41 (s, 6H), 1.12 (s, 2H). ¹³CNMR (100 MHz, DMSO-d₆) δ 146.2, 137.6, 131.4, 128.5, 124.8, 123.9, 122.7, 122.6, 119.2, 116.9, 116.8, 115.8, 52.7, 37.7, 26.7, 24.9. HRMS(ESI), calculated for C₂₀H₂₃N₄⁺ [M − I]⁺ 319.1917, found 319.1917. |
| Example 37 | formula (I-58) | ¹HNMR (400 MHz, MeOH-d₄) δ 8.50 (d, J = 8.5 Hz, 1H), 8.41 (d, J = 8.5 Hz, |
| | | 1H), 8.19 (d, J = 8.9 Hz, 1H), 7.99-7.94 (m, 1H), 7.76 (d, J = 8.4 Hz, 1H), 7.68- |
| | 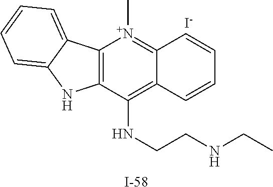 | 7.62 (m, 2H), 7.37-7.31 (m, 1H), 4.54 (s, 3H), 4.24-4.17 (m, 2H), 3.23-3.19 (m, 2H), 2.83 (q, J = 7.2 Hz, 2H), 1.16 (t, J = 7.2 Hz, 3H). ¹³CNMR (100 MHz, MeOH-d₄) δ 143.4, 137.4, 136.1, 132.1, 130.5, 124.9, 124.1, 123.6, 123.1, 120.7, 116.7, 115.5, 115.2, 113.3, 45.2, 43.7, 37.2, 13.2. HRMS(ESI), calculated for C₂₀H₂₃N₄⁺ [M − I]⁺ 319.1917, found 319.1917. |
| Example 38 | formula (I-59) | ¹HNMR (400 MHz, MeOH-d₄) δ 8.48 (d, J = 8.4 Hz, 1H), 8.38 (d, J = 8.5 Hz, |
| | | 1H), 8.16 (d, J = 8.9 Hz, 1H), 8.00-7.92 (m, 1H), 7.75 (d, J = 8.4 Hz, 1H), |
| | 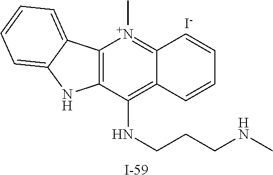 | 7.70-7.60 (m, 2H), 7.32 (t, J = 7.7 Hz, 1H), 4.49 (s, 3H), 4.20 (t, J = 6.3 Hz, 2H), 2.87 (t, J = 6.0 Hz, 2H), 2.47 (s, 3H), 2.20-2.09 (m, 2H). ¹³CNMR (100 MHz, MeOH-d₄) δ 144.5, 144.0, 137.3, 136.1, 132.0, 130.3, 124.0, 123.5, 123.0, 120.5, 116.6, 115.3, 114.9, 113.4, 100.0, 46.8, 42.9, 37.1, 34.0, 28.7. HRMS(ESI), calculated for C₂₀H₂₃N₄⁺ [M − I]⁺ 319.1917, found 319.1917. |
| Example 39 | formula (I-60) | ¹HNMR (400 MHz, DMSO-d₆) δ 10.76 (s, 1H), 8.57 (d, J = 8.4 Hz, 1H), 8.36 (d, |
| | | J = 8.9 Hz, 1H), 8.19 (d, J = 8.4 Hz, 1H), 8.07-8.00 (m, 1H), 7.86-7.71 (m, 3H), |
| | 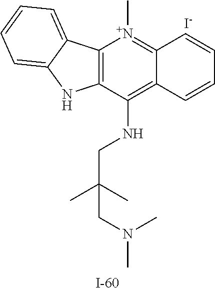 | 7.42-7.34 (m, 1H), 4.60 (s, 3H), 4. 15 (s, 2H), 3.36 (s, 1H), 2.62 (s, 2H), 2.46 (s, 6H), 1.15 (s,6H). ¹³CNMR (100 MHz, DMSO-d₆) δ 144.5, 143.0, 137.7, 135.8, 132.8, 130.9, 125.0, 124.8, 123.0, 121.2, 118.1, 116.7, 115.6, 115.0, 114.0, 100.0, 69.9, 57.3, 48.2, 38.4, 35.4, 25.5. HRMS(ESI), calculated for C₂₃H₂₉N₄⁺ [M − I]⁺ 361.2387, found 361.2385. |
| Example 40 | formula (I-61) | ¹HNMR (400 MHz, DMSO-d₆) δ 8.42 (d, J = 8.3 Hz, 2H), 8.14 (d, J = 8.7 Hz, 1 |
| | | H), 7.94-7.88 (m, 1H), 7.81 (d, J = 8.2 Hz, 1H), 7.66-7.60 (m, 1H), 7.55-7.51 |
| | 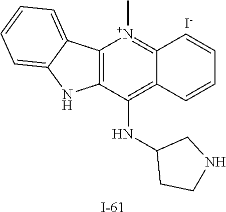 | (m, 1H), 7.34-7.26 (m, 1H), 4.46 (s, 3H), 3.91 (d, J = 10.2 Hz, 1H), 3.68 (s, 1H), 2.15 (s, 1H), 1.91(d, J = 5.2 Hz, 2H), 1.21 (s, 1H), 0.85 (s, 1H). ¹³CNMR (100 MHz, DMSO-d₆) δ 147.5, 142.5, 139.1, 134.4, 132.2, 129.9, 128.1, 124.1, 122.0, 120.8, 119.6, 116.8, 116.5, 114.7, 114.2, 63.9, 53.8, 50.8, 38.5, 33.8. HRMS(ESI), calculated for C₂₀H₂₁N₄⁺ [M − I]⁺ 317.1761, found 317.1761. |
| Example 41 | formula (I-62) | ¹HNMR (400 MHz, DMSO-d₆) δ 8.74 (d, J = 8.3 Hz, 1H), 8.52 (d, J = 7.9 Hz, 1 |
| | | H), 8.33 (d, J = 8.8 Hz, 1H), 8.00 (t, 1H), 7.86 (d, J = 8.3 Hz, 1H), 7.73-7.62 |
| | 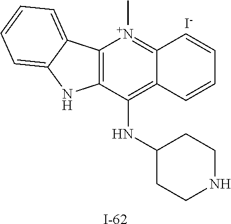 | (m, 2H), 7.31 (t, 1H), 5.05 (s, 1H), 4.60 (s, 3H), 3.49-3.39 (m, 1H), 3.33 (d, J = 11.9 Hz, 2H), 3.10 (t, J = 11.7 Hz, 2H), 2.22-2.13 (m, 2H), 1.96- 1.88 (m, 2H). HRMS(ESI), calculated for C₂₁H₂₃N₄⁺ [M − I]⁺ 331.1917, found 331.1919. |
| Example 42 | formula (I-63) | ¹HNMR (400 MHz, DMSO-d₆) δ 8.77 (d, J = 8.5 Hz, 1H), 8.53 (d, J = 8.4 Hz, 1 |
| | | H), 8.33 (d, J = 8.9 Hz, 1H), 8.03-7.97 (m, 1H), 7.85 (d, J = 8.4 Hz, 1H), 7.68 |
| | 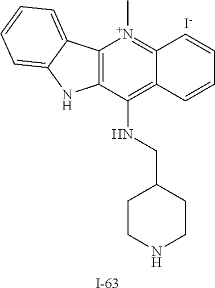 | (t, J = 6.9 Hz, 2H), 7.32 (t, J = 7.6 Hz, 1H), 4.60 (s, 3H), 4.15 (d, J = 6.8 Hz, 2H), 3.24 (d, J = 12.4 Hz, 2H), 2.76 (t, J = 11.7 Hz, 2H), 2.13 (s, 1H), 1.95 (d, J = 12.3 Hz, 2H), 1.53-1.40 (m, 2H). ¹³CNMR (100 MHz, DMSO-d₆) δ 144.3, 137.5, 135.8, 132.4, 130.3, 124.6, 124.5, 124.1, 120.6, 118.8, 118.2, 116.6, 115.7, 115.1, 114.9, 50.4, 43.6, 38.4, 35.1, 27.1. HRMS(ESI), calculated for C₂₂H₂₅N₄⁺ [M − I]⁺ 345.2074, found 345.2073. |
| Example 43 | formula (I-64) | ¹HNMR (400 MHz, DMSO-d₆) δ 8.92 (s, 1H), 8.72 (d, J = 8.4 Hz, 1H), 8.54 (d, |
| | | J = 8.4 Hz, 1H), 8.35 (d, J = 8.9 Hz, 1H), 8.07-7.98 (m, 1H), 7.88 (d, J = 8.3 Hz, |
| | 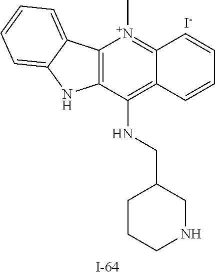 | 1H), 7.79-7.67 (m, 2H), 7.43-7.33 (m, 1H), 4.60 (s, 3H), 4.20 (s, 1H), 4.11-3.90 (m, 2H), 3.18 (d, J = 11.9 Hz, 2H), 2.92-2.79 (m, 2H), 2.32 (s, 1H), 2.00-1.79 (m, 2H), 1.61-1.34 (m, 2H). ¹³CNMR (100 MHz, DMSO-d₆) δ 144.1, 137.5, 136.2, 132.7, 132.7, 131.0, 124.8, 124.5, 124.4, 121.1, 117.9, 115.7, 115.0, 114.4, 100.0, 47.9, 46.3, 44.0, 38.6, 34.5, 26.1, 21.6. HRMS(ESI), calculated for C₂₂H₂₅N₄⁺ [M − I]⁺ 345.2074, found 345.2074. |
| Example 44 | formula (I-65) | ¹HNMR (400 MHz, DMSO-d₆) δ 11.90 (s, 1H), 8.74 (d, J = 8.1 Hz, 1H), 8.55 (d, |
| | | J = 8.1 Hz, 1H), 8.36 (d, J = 8.3 Hz, 2H), 8.04 (t, J = 7.5 Hz, 1H), 7.93 (d, J = |
| | 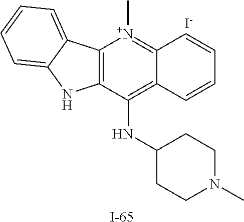 | 8.1 Hz, 1H), 7.81-7.68 (m, 2H), 7.39 (t, J = 7.2 Hz, 1H), 4.86 (s, 1H), 4.62 (s, 3H), 3.28-3. 15 (m, 4H), 2.77 (s, 3H), 2.29-2.08 (m, 4H). ¹³CNMR (100 MHz, DMSO-d₆) δ 143.4, 143.3, 137.5, 136.7, 132.9, 131.4, 125.0, 124.6, 121.5, 117.9, 117.4, 116.2, 115.2, 114.1, 54.9, 52.6, 50.1, 38.8, 30.40 HRMS(ESI), calculated for C₂₂H₂₅N₄⁺ [M − I]⁺ 345.2074, found 345.2074. |
| Example 45 | formula (I-66) | ¹HNMR (400 MHz, DMSO-d₆) δ 11.83 (s, 1H), 8.86 (s, 1H), 8.77 (d, J = 8.6 Hz, |
| | | 1H), 8.60 (d, J = 8.6 Hz, 1H), 8.39 (d, J = 9.0 Hz, 1H), 8.05 (t, 1H), 7.89 (d, |
| | 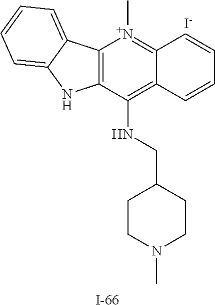 | J = 8.4 Hz, 1H), 7.81-7.72 (m, 2H), 7.41 (t, J = 7.7 Hz, 1H), 4.65 (s, 3H), 4.07 (s, 2H), 3.11 (s, 5H), 2.70 (s, 3H), 2.16-1.96 (m, 3H), 1.63-1.47 (m, 2H). ¹³CNMR (100 MHz, DMSO-d₆) δ 144.1, 143.3, 137.7, 136.3, 132.9, 131.2, 124.9, 124.7, 124.4, 121.4, 118.0, 117.0, 115.8, 115.1, 114.1, 54.9, 49.9, 43.1, 38.6, 34.0, 27.2. HRMS(ESI), calculated for C₂₃H₂₇N₄⁺ [M − I]⁺ 359.2230, found 359.2229. |
| Example 46 | formula (I-67) | ¹HNMR (400 MHz, DMSO-d₆) δ 8.68 (d, J = 8.5 Hz, 1H), 8.53 (d, J = 8.4 Hz, 1 |
| | | H), 8.32 (d, J = 8.9 Hz, 1H), 8.00 (t, 1H), 7.79 (d, J = 8.3 Hz, 1H), 7.70 (t, J = |
| | 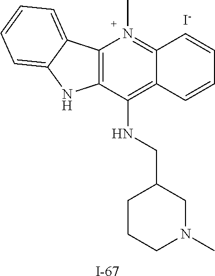 | 7.6 Hz, 2H), 7.35 (t, J = 7.6 Hz, 1H), 4.57 (s, 3H), 4.50 (s, 1H), 3.69 (d, J = 15.0 Hz, 1H), 2.47-2.33 (m, 2H), 2.31-2.22 (m, 1H), 2.08 (s, 3H), 1.86-1.78 (m, 1H), 1.76-1.67 (m, 1H), 1.66-1.56 (m, 1H), 1.54-1.43 (m, 1H), 1.27- 1.18 (m, 1H), 0.88-0.79 (m, 1H). ¹³CNMR (100 MHz, DMSO-d₆) δ 144.8, 143.9, 137.5, 136.3, 132.6, 130.8, 124.7, 124.4, 124.1, 120.9, 117.8, 115.6, 115.5, 114.4, 55.5, 47.8, 45.8, 38.4, 35.1, 26.3, 22.4, 14.4. HRMS(ESI), calculated for C₂₃H₂₇N₄⁺ [M − I]⁺ 359.2230, found 359.2231. |
| Example 47 | formula (I-68) | ¹HNMR (400 MHz, DMSO-d₆) δ 8.89 (s, 1H), 8.65 (d, J = 8.3 Hz, 1H), 8.53 (d, |
| | | J = 8.2 Hz, 1H), 8.33 (d, J = 8.8 Hz, 1H), 8.06-7.98 (m, 1H), 7.88 (d, J = 8.2 Hz, |
| | 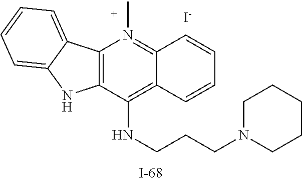 | 1H), 7.77-7.67 (m, 2H), 7.42-7.33 (m, 1H), 4.56 (s, 3H), 4.18 (s, 2H), 3.14- 2.78 (m, 6H), 2.17 (s, 2H), 1.67 (s, 4H), 1.52 (s, 2H). ¹³CNMR (100 MHz, DMSO-d₆) δ 143.4, 142.7, 137.0, 135.5, 132.3, 130.6, 124.3, 124.0, 123.9, 120.8, 117.4, 116.2, 115.1, 114.4, 113.5, 54.2, 52.7, 43.1, 38.1, 24.7, 23.3, 21.9. HRMS(ESI), calculated for C₂₄H₂₉N₄⁺ [M − I]⁺ 373.2387, found 373.2383. |

The preferred embodiments of the invention are described in detail above, however, the invention is not limited to this. Within the scope of the technical conception of the invention, a variety of simple variants of the technical scheme of the invention can be carried out, including the combination of each technical feature in any other suitable way. These simple variants and combinations shall also be regarded as the contents disclosed by the invention and belong to the scope of protection of the invention.

1.-12. (canceled)

13. A cryptolepine salt derivative with a chemical structure shown in formula (I), 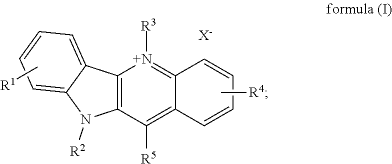
wherein
R¹ is selected from at least one group consisting of hydrogen, C1-C6 alkyl, C1-C6 alkoxy, halogen, and cyano;
R² is selected from at least one group consisting of hydrogen and protecting group;
R³ is selected from at least one group consisting of substituted or unsubstituted C1-C6 alkyl, and C6-C20 aromatic group; wherein substituent of substituted C1-C6 alkyl is selected from at least one group consisting of C3-C6 cycloalkyl;
R⁴ is selected from at least one group consisting of hydrogen, C1-C6 alkyl, C1-C6 alkoxy, halogen, C1-C6 fluoroalkyl, and C2-C5 ester group;
R⁵ is selected from at least one group consisting of hydrogen, and —NHR; wherein R is selected from at least one group consisting of substituted or unsubstituted C1-C20 alkyl, C3-C6 cycloalkyl, C3-C12 azacyclic alkyl, and C3-C12 azacyclic alkyl substituted with C1-C6 alkyl; wherein substituent of substituted C1-C20 alkyl is selected from at least one group consisting of C3-C6 cycloalkyl, amino, amino substituted with C1-C12 alkyl, C3-C12 cycloamine group, C3-C12 azacyclic alkyl, and C3-C12 azacyclic alkyl substituted with C1-C6 alkyl;
X⁻ is selected from salt-forming anion.

14. The cryptolepine salt derivative according to claim 13, wherein R¹ is selected from at least one group consisting of hydrogen, C1-C3 alkyl, C1-C3 alkoxy, halogen, and cyano;
R² is selected from at least one group consisting of hydrogen, p-toluenesulfonyl, benzenesulfonyl, benzyloxycarbonyl, tert-butoxycarbonyl and benzyl;
R³ is selected from at least one group consisting of substituted or unsubstituted C1-C6 alkyl, and C6-C12 aromatic group; wherein substituent of substituted C1-C6 alkyl is selected from at least one group consisting of C3-C6 cycloalkyl;
R⁴ is selected from at least one group consisting of hydrogen, C1-C3 alkyl, C1-C3 alkoxy, halogen, C1-C3 perfluoroalkyl, and C2-C5 ester group;
R⁵ is selected from at least one group consisting of hydrogen and —NHR; wherein R is selected from at least one group consisting of substituted or unsubstituted C1-C12 alkyl, C3-C6 cycloalkyl, C3-C6 azacyclic alkyl, and C3-C6 azacyclic alkyl substituted with C1-C3 alkyl; wherein substituent of substituted C1-C12 alkyl is selected from at least one group consisting of C3-C6 cycloalkyl, amino, amino substituted with C1-C6 alkyl, C3-C6 cycloamine group, C3-C6 azacyclic alkyl, and C3-C6 azacyclic alkyl substituted with C1-C3 alkyl;
X⁻ is selected from at least one group consisting of Cl⁻, Br⁻, I⁻, CH₃COO⁻, NO₃⁻, HSO₄⁻, H₂PO₄⁻, BF₄⁻, and SbF₆⁻.

15. The cryptolepine salt derivative according to claim 14, wherein,
situation 1: when X⁻ is Cl⁻, it is satisfied that:
R¹ is selected from at least one group consisting of hydrogen, methyl, methoxy, F, Cl, Br, I, and cyano; R² is selected from at least one group consisting of hydrogen, p-toluenesulfonyl, and benzenesulfonyl; R³ is selected from at least one group consisting of methyl, 2,2-dimethylpropyl, cyclopropylmethyl, cyclohexylmethyl, and benzyl; R⁴ is selected from at least one group consisting of hydrogen, methyl, methoxy, F, Cl, Br, I, trifluoromethyl, and methyl formate group; R⁵ is hydrogen;
situation 2: when X⁻ is I⁻, it is satisfied that:
R¹, R², R⁴ is hydrogen, R³ is methyl, R⁵ is selected from at least one of —NHR, wherein R is selected from at least one group consisting of substituted or unsubstituted C1-C12 alkyl, C3-C6 cycloalkyl, C3-C6 azacyclic alkyl, and C3-C6 azacyclic alkyl substituted with C1-C3 alkyl; wherein substituent of substituted C1-C12 alkyl is selected from at least one group consisting of C3-C6 cycloalkyl, amino, amino substituted with C1-C6 alkyl, C3-C6 cycloamine group, C3-C6 azacyclic alkyl, and C3-C6 azacyclic alkyl substituted with C1-C3 alkyl.

16. The cryptolepine salt derivative according to claim 15, wherein, referring to the situation 2, when X⁻ is I⁻, it is satisfied that: R¹, R², R⁴ is hydrogen, R³ is methyl, R⁵ is selected from —NHR, wherein R comprises at least one group consisting of linear or branched C3-C12 alkyl, C3-C6 cycloalkyl, methyl substituted with C3-C6 cycloalkyl, propyl substituted with C3-C6 cycloalkyl, substituted or unsubstituted C2-C6 alkylamino, tetrahydropyrrole group, piperidyl, piperidyl methyl, n-methylpiperidyl, and n-methyl piperidine methyl; wherein substituent of substituted C2-C6 alkylamino is selected from at least one group consisting of methyl, ethyl, and

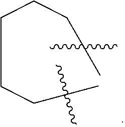 [SMILES: *CCCCC*]

17. The cryptolepine salt derivative according to claim 15, wherein R is selected from at least one group consisting of n-propyl, n-amyl, isoamyl, n-dodecyl, 2-ethylhexyl, cyclopropyl, cyclopentyl, cyclohexyl, cyclopropylmethyl, cycloamylmethyl, cyclohexylmethyl, cyclopropylethyl, cycloamylethyl, cyclohexylethyl, cyclopropylpropyl, cycloamylpropyl, cyclohexyl propyl, aminomethyl, aminoethyl, aminopropyl, aminobutyl, 2-aminopropyl, 2-methyl-2-amino-propyl, —CH₂CH₂—NH—CH₂CH₃, —CH₂CH₂CH₂—NH—CH₃, —CH₂C(CH₃)₂—CH₂—N(CH₃)₂,

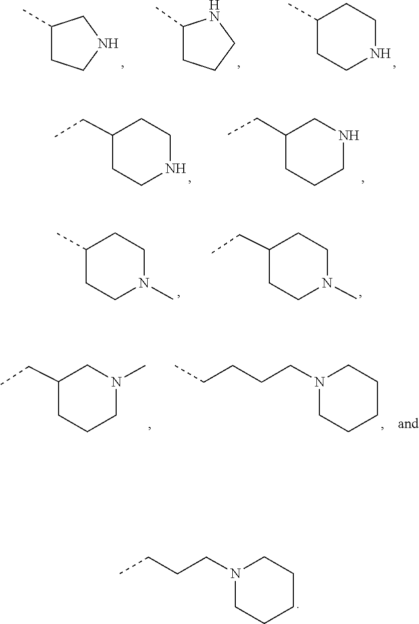

18. The cryptolepine salt derivative according to claim 13, wherein the cryptolepine salt derivative comprises at least one of the compounds shown in formula (I-1) to formula (I-71):

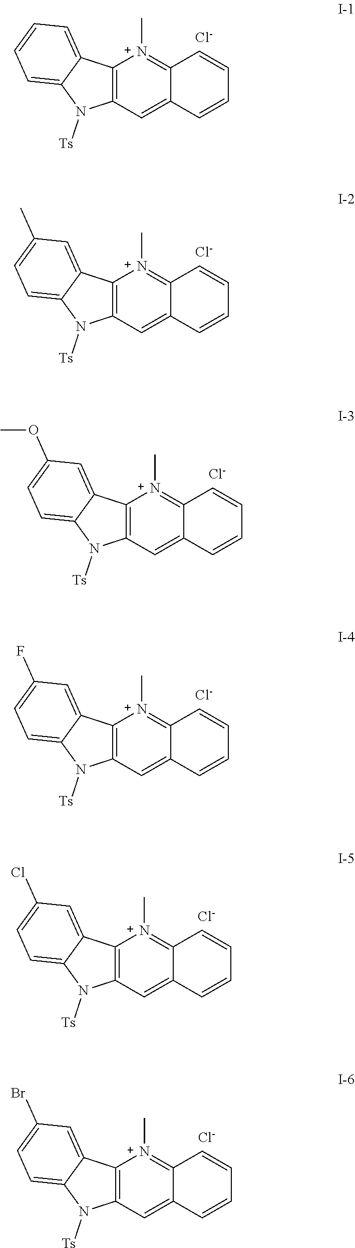

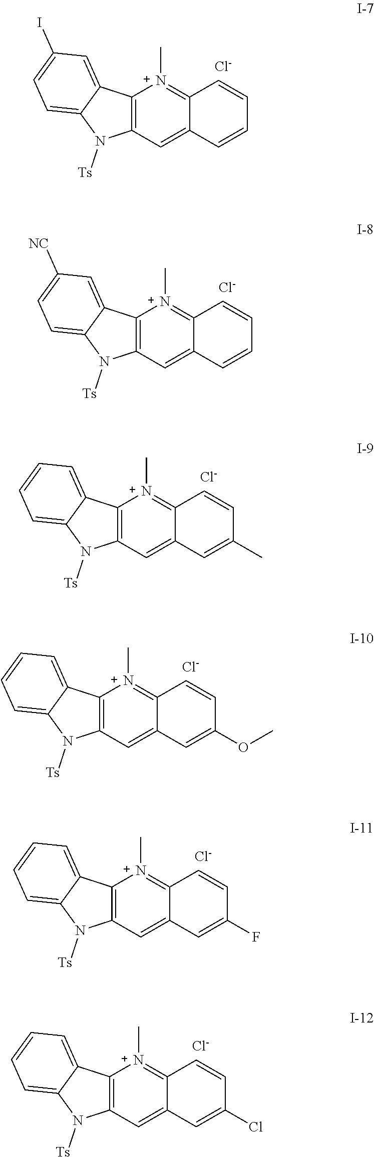

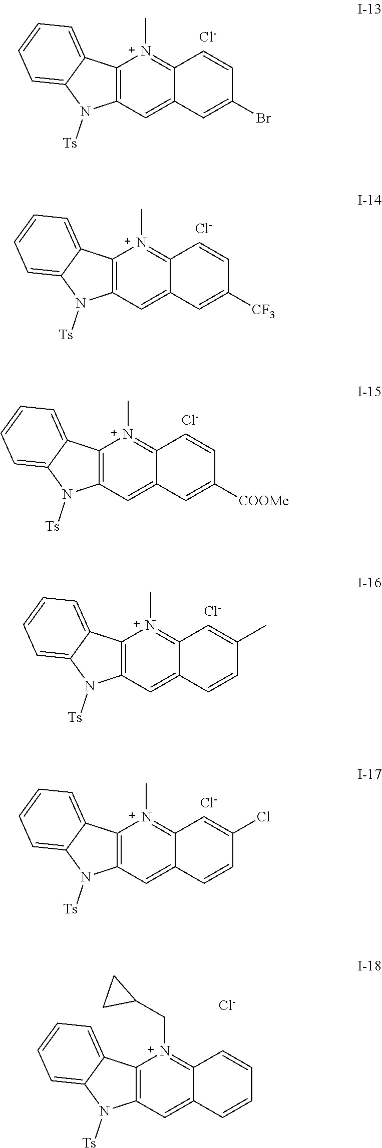

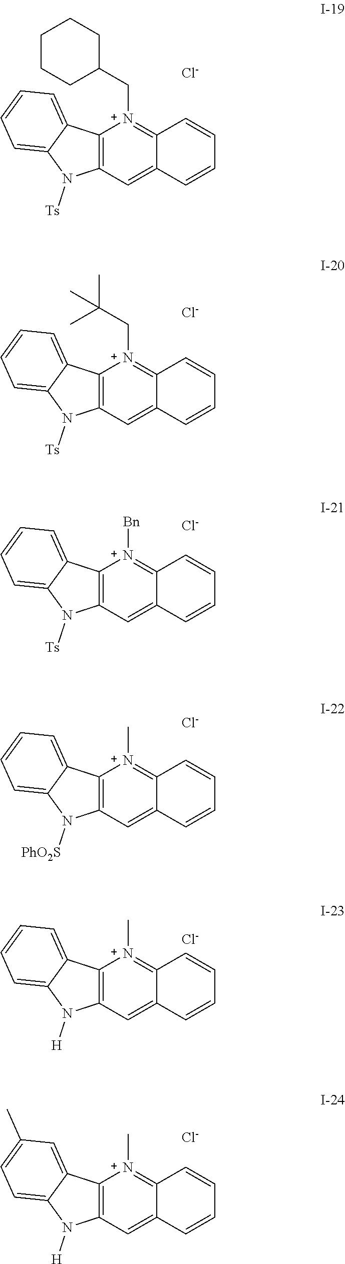

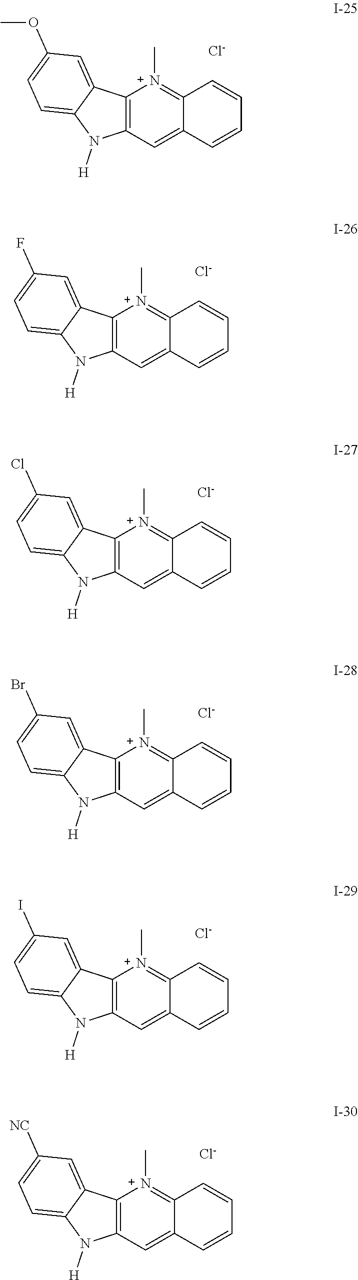

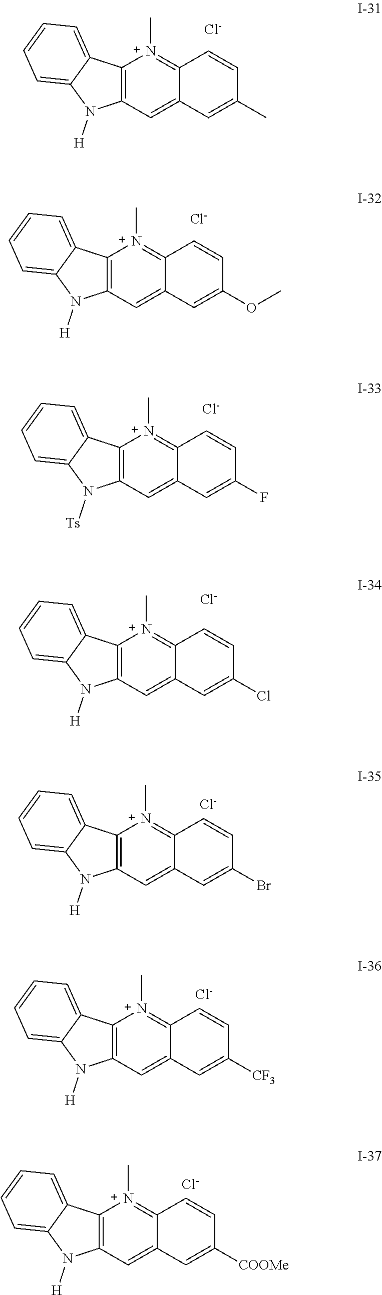

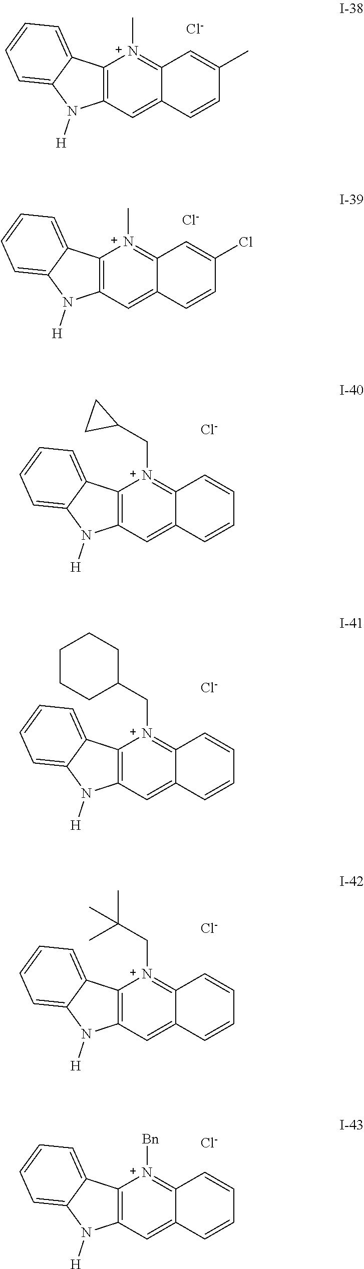

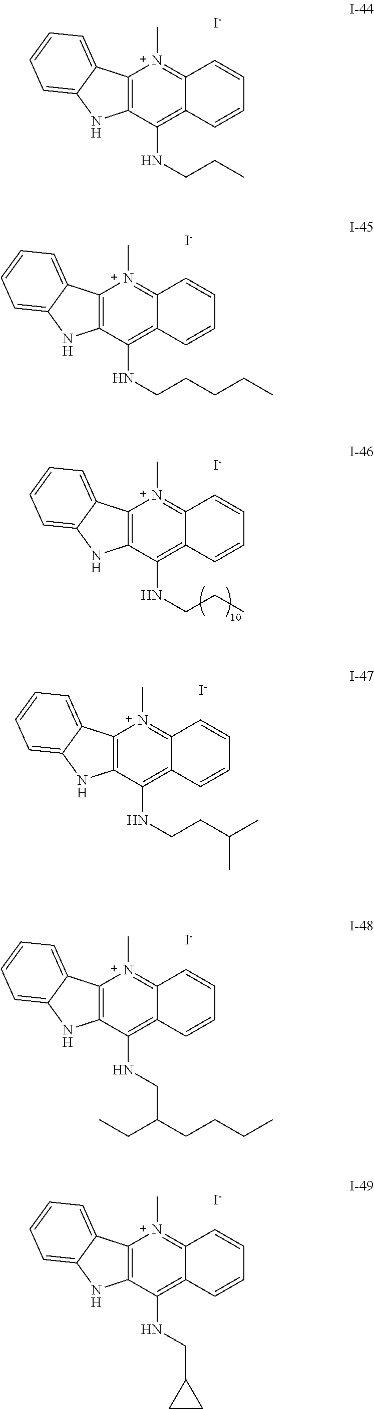

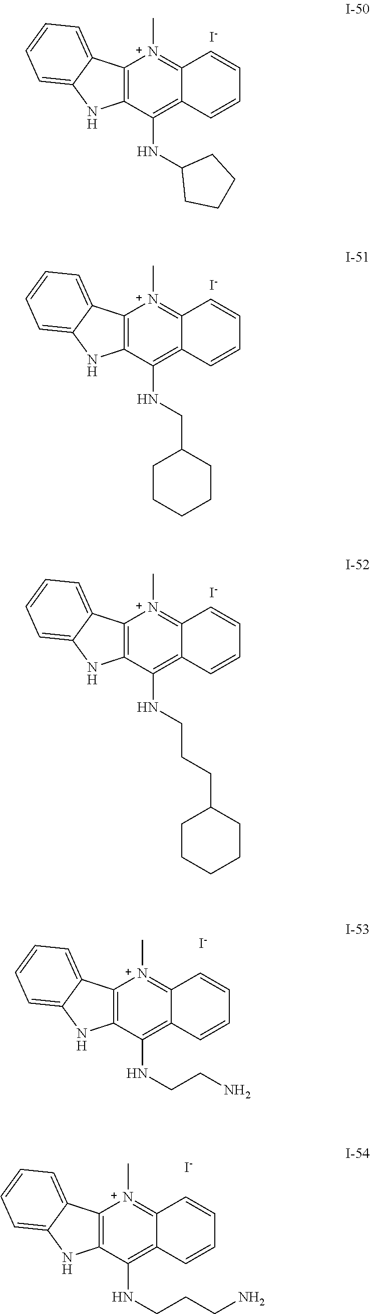

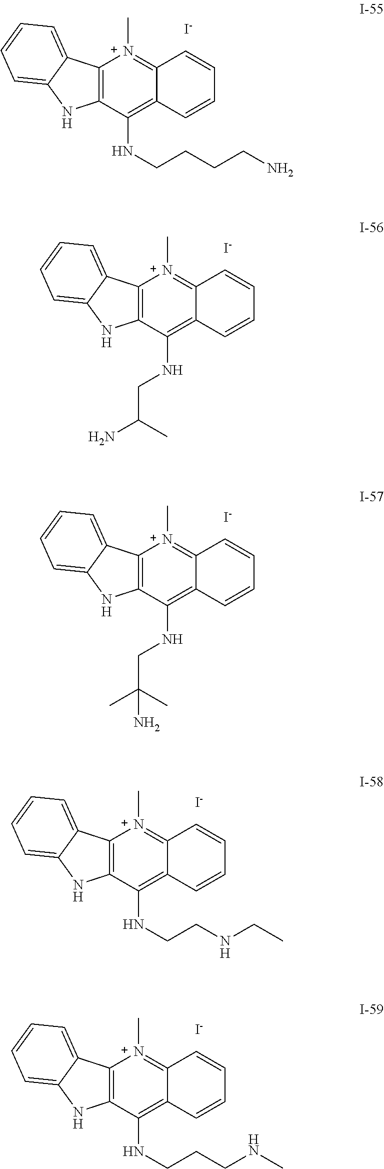

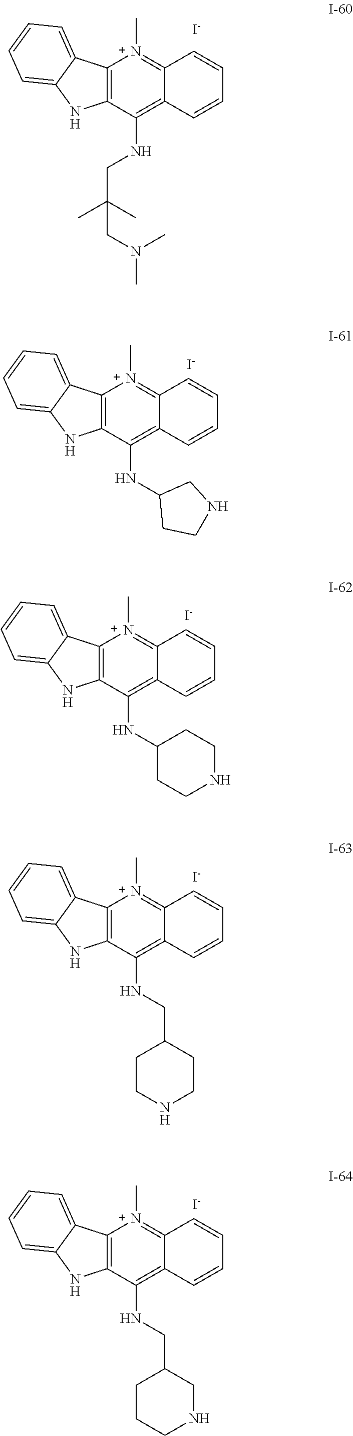

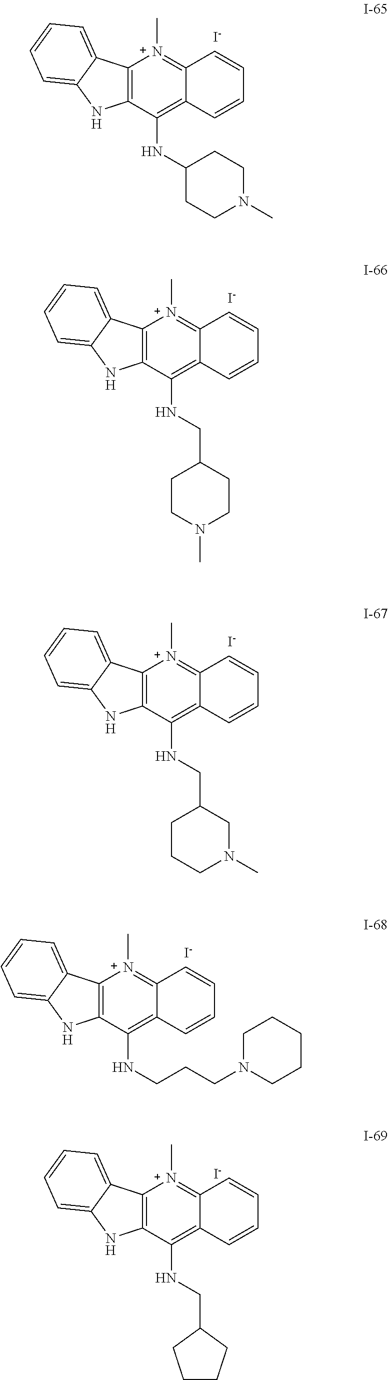

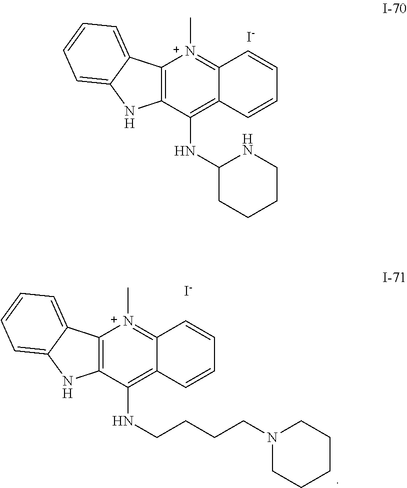 [SMILES: C[n+]1c2ccccc2c(NC2CCCCN2)c2[nH]c3ccccc3c21 ; [I-] ; C[n+]1c2ccccc2c(NCCCCN2CCCCC2)c2[nH]c3ccccc3c21 ; [I-]]

19. The cryptolepine salt derivative according to claim 18, wherein the cryptolepine salt derivative comprises at least one of the following compounds:

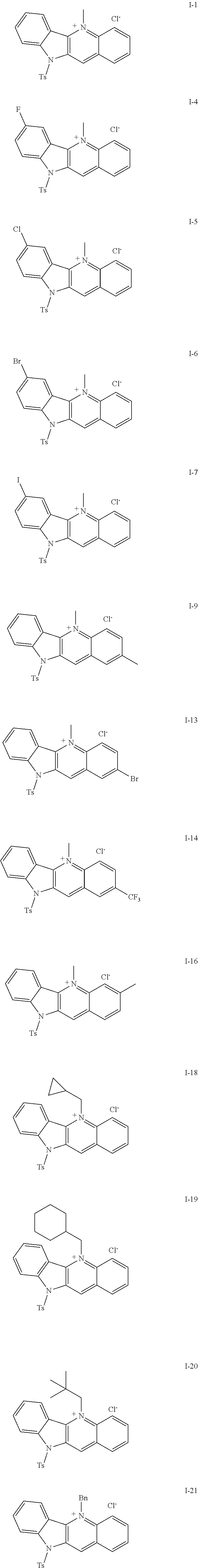

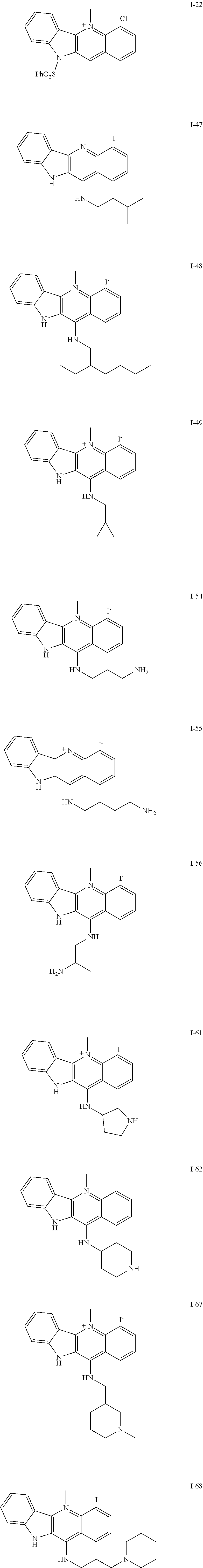

20. A preparation method of the cryptolepine salt derivative according to claim 13, comprising:
method (1): when R⁵ is hydrogen, in presence of catalyst and formylation reagent, making compound A 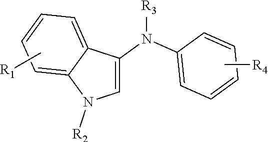
undergo electrophilic substitution reaction, thus to obtain the cryptolepine salt derivative;
method (2): when R⁵ is selected from —NHR, making compound B 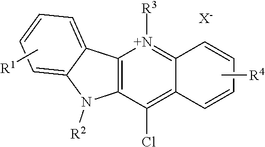
undergo nucleophilic substitution reaction with RNH₂ to obtain the cryptolepine salt derivative.

21. The preparation method according to claim 20, comprising:
method (1): when R⁵ is hydrogen, in presence of catalyst and formylation reagent, making compound A 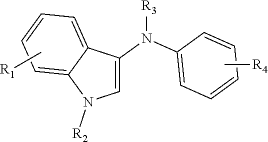
undergo electrophilic substitution reaction with imide salt, thus to obtain the cryptolepine salt derivative;
method (2): when R⁵ is selected from —NHR, making compound B 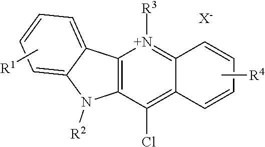
undergo nucleophilic substitution reaction with RNH₂ under a condition of heating reflux by using ethyl acetate as solvent to obtain the cryptolepine salt derivative.

22. The preparation method according to claim 21, in the method (1), the catalyst is selected from at least one of POCl₃, SOCl₂, ZnCl₂ and COCl₂;
and/or, the formylation reagent is selected from at least one of N-substituted formamides;
and/or, molar ratio of the compound A and the catalyst is 1:1.2-3;
and/or, the compound A is provided in solution when added to reaction system, wherein concentration of the compound A is 0.2-1 mmol/mL;
and/or, temperature of the electrophilic substitution reaction is-20° C. to 30° C., time of the electrophilic substitution reaction is 0.5-4 h;
and/or, the imide salt is an intermediate 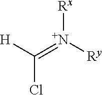
obtained by a reaction of the catalyst and the formylation reagent, wherein R^{x} and R^{y} are two substituents on the N position of N-substituted formamide, respectively.

23. The preparation method according to claim 22, in the method (1), the catalyst is selected from at least one of POCl₃ and SOCl₂;
and/or, the formylation reagent is selected from at least one of N,N-Dimethylformamide, N-Methyl-N-phenylformamide, N,N-Dipropylformamide, and N-ethyl-N-methylformamide;
and/or, molar ratio of the compound A and the catalyst is 1:1.5-2.5;
and/or, the compound A is provided in solution when added to reaction system, wherein concentration of the compound A is 0.4-0.6 mmol/mL;
and/or, temperature of the electrophilic substitution reaction is −16° C. to 25° C., time of the electrophilic substitution reaction is 1-2 h.

24. The preparation method according to claim 23, in the method (1), the formylation reagent is selected from at least one of N,N-Dimethylformamide and N-Methyl-N-phenylformamide.

25. The preparation method according to claim 21, in the method (2), temperature of the heating reflux is 90-150° C., time of the heating reflux is 10-24 h.

26. The preparation method according to claim 25, in the method (2), temperature of the heating reflux is 110-130° C., time of the heating reflux is 15-17 h.

27. The preparation method according to claim 21, in the method (2), molar ratio of the compound B to RNH₂ is 1:1-5.

28. The preparation method according to claim 27, in the method (2), molar ratio of the compound B to RNH₂ is 1:1-3.

29. A method of preventing and controlling plant virus or sterilizing comprising administering the cryptolepine salt derivative according to claim 1.
